(19) United States
(12) Patent Application Publication
(10) Pub. No.: US 2014/0221837 A1
(43) Pub. Date: Aug. 7, 2014

(54) ULTRASOUND APPARATUS AND CONTROL METHOD THEREOF

(71) Applicant: SAMSUNG ELECTRONICS CO., LTD., Suwon-si (KR)

(72) Inventors: Jin Soo PARK, Suwon-si (KR); Hyung Sok YEO, Hwaseong-si (KR); Kwang Hyeon LEE, Seoul (KR)

(73) Assignee: SAMSUNG ELECTRONICS CO., LTD., Suwon-si (KR)

(21) Appl. No.: 14/169,604

(22) Filed: Jan. 31, 2014

(30) Foreign Application Priority Data
Feb. 1, 2013 (KR) 10-2013-0012038

(51) Int. Cl. A61B 8/08; A61M 5/46

(52) U.S. Cl. CPC A61B 8/0858; A61B 8/085; A61M 5/46
USPC 600/449; 604/117; 604/506

(57) ABSTRACT

Disclosed are an ultrasound apparatus and a control method thereof, in which the thickness of subcutaneous tissue is measured using an ultrasound probe or vital data regarding a subject is acquired to select an appropriate injection needle and determine suitability of injection to a corresponding body region, which ensures safe and effective drug administration. In the ultrasound apparatus and the control method thereof, a map containing results of selection of an appropriate injection needle and determination of injection suitability is generated and provided to a patient, which ensures safe self-administration by the patient without a hospital visit. The ultrasound apparatus includes a data acquirer to acquire ultrasound data regarding a subject, a thickness calculator to calculate the thickness of subcutaneous tissue inside the subject based on the ultrasound data, and a needle selector to select a needle corresponding to the calculated thickness of subcutaneous tissue.

### CROSS-REFERENCE TO RELATED APPLICATION

This application claims the benefit of Korean Patent Applications No. 2013-0012038, filed on Feb. 1, 2013 in the Korean Intellectual Property Office, the disclosure of which is incorporated herein by reference.

### BACKGROUND

1. Field

Exemplary embodiments relate to an ultrasound apparatus that provides data regarding drug injection, and a control method thereof.

2. Description of the Related Art

Administration of drugs to patients includes oral administration and injection. Drug administration is determined in consideration of purposes of drug administration, patient health factors, and the like. In the case of injection, a body region in which injection is performed may be determined based on purposes of drug administration, patient health factors, and the like.

Diabetic patients need periodic administration of insulin, which is performed via injection. Insulin is most commonly subcutaneously injected and is mainly injected into the abdomen, arm, thigh, hip, the like, and other parts of the body.

As subcutaneous tissue is located between the skin and the muscles, an excessively short injection needle may cause insulin to be administered to the skin, whereas an excessively long injection needle may cause insulin to be administered to the muscles. Therefore, although it may be necessary to select an appropriate injection needle length according to the thickness of subcutaneous tissue, impossibility of observing the thickness of subcutaneous tissue makes it difficult to achieve safe and effective supply of insulin.

Moreover, it may be necessary to protect specific body regions having relatively hard skin, varicose veins or damaged blood vessels from injection, but observing these regions may likewise be difficult.

### SUMMARY

It is one aspect of an exemplary embodiment to provide an ultrasound apparatus and a control method thereof, in which the thickness of subcutaneous tissue is measured using an ultrasound probe or vital data regarding a subject is acquired to select an appropriate injection needle and determine suitability of injection to a corresponding body region, which ensures safe and effective drug administration.

It is another aspect of an exemplary embodiment to provide an ultrasound apparatus and a control method thereof, in which a map containing results of selection of an appropriate injection needle and determination of injection suitability is generated and provided to a patient, which ensures safe self-administration by the patient without a hospital visit.

Additional aspects will be set forth in part in the description which follows and, in part, will be obvious from the description, or may be learned by practice of the invention.

In accordance with one aspect, an ultrasound apparatus includes a data acquirer to acquire ultrasound data regarding a subject, a thickness calculator to calculate the thickness of subcutaneous tissue inside the subject based on the ultrasound data, and a needle selector to select a needle corresponding to the calculated thickness of subcutaneous tissue.

The data acquirer may include an ultrasound probe to transmit ultrasound to the subject and receive ultrasound reflected from the subject.

The needle selector may select a needle having a length corresponding to the calculated thickness of subcutaneous tissue.

The needle selector may select a needle having a length to ensure insertion of a needle tip into the subcutaneous tissue.

The ultrasound apparatus may further include a display device or a speaker to suggest the selected needle.

The ultrasound apparatus may further include a map generator to generate a needle length map by matching a needle selected per region of the subject to a corresponding region.

The ultrasound apparatus may further include a vital data acquirer to acquire vital data regarding the subject based on the ultrasound data, and an injection region determiner to determine injection suitability based on the acquired vital data regarding the subject.

The vital data regarding the subject may be at least one selected from the group including hardness of the skin, the presence of varicose veins, and the presence of damaged blood vessels.

The injection region determiner may determine that injection is not suitable if hardness of the skin reaches or exceeds a predetermined reference value, if varicose veins are present, or if damaged blood vessels are present.

The ultrasound apparatus may further include a display device or a speaker to output the result of determination of injection suitability.

The ultrasound apparatus may further include a map generator to generate an injection map by matching a needle selected per region of the subject and the result of determination of injection suitability to a corresponding region.

The ultrasound apparatus may further include a syringe coupled to the ultrasound probe, and a syringe controller to control a needle length of the syringe according to the needle selected by the needle selector.

In accordance with another aspect of an exemplary embodiment, an ultrasound apparatus includes a data acquirer to acquire ultrasound data regarding the subject, a vital data acquirer to acquire vital data regarding the subject based on the ultrasound data, and an injection region determiner to determine injection suitability based on the acquired vital data regarding the subject.

The vital data regarding the subject may be at least one selected from the group including hardness of the skin, the presence of varicose veins, and the presence of damaged blood vessels.

The injection region determiner may determine that injection is not suitable if hardness of the skin reaches or exceeds a predetermined reference value, if varicose veins are present, or if damaged blood vessels are present.

The ultrasound apparatus may further include a display device or a speaker to output the result of determination of injection suitability.

The ultrasound apparatus may further include a map generator to generate an injection region map by matching the result of determination of injection suitability to each region of the subject.

The ultrasound apparatus may further include a syringe coupled to the probe, and a syringe controller to control the syringe such that a needle of the syringe is inserted into the subject by the length of the selected needle.

In accordance with another aspect of an exemplary embodiment, a control method of an ultrasound apparatus, includes acquiring ultrasound data regarding a subject, measuring the thickness of subcutaneous tissue of the subject based on the acquired ultrasound data, and selecting a needle corresponding to the measured thickness of subcutaneous tissue.

Selection of the needle may include selecting a needle having a length corresponding to the measured thickness of subcutaneous tissue.

Selection of the needle may include selecting a needle having a length to ensure insertion of a needle tip into the subcutaneous tissue.

The control method may further include generating a needle length map by matching a needle selected per region of the subject to a corresponding region.

The control method may further include acquiring vital data regarding the subject based on the acquired ultrasound data, and determining suitability of injection to a corresponding region based on the acquired vital data regarding the subject.

The vital data regarding the subject may be at least one selected from the group including hardness of the skin, the presence of varicose veins, and the presence of damaged blood vessels.

Determination of injection suitability may include determining that injection is not suitable if hardness of the skin reaches or exceeds a predetermined reference value, if varicose veins are present, or if damaged blood vessels are present.

The control method may further include visibly or audibly outputting the result of determination of injection suitability.

The control method may further include generating an injection map by matching a needle selected per region of the subject and the result of determination of injection suitability to a corresponding region.

The control method may further include automatically controlling a needle length of the syringe according to the length of the selected needle.

The control method may further include visibly or audibly suggesting the selected needle.

In accordance with a further aspect of an exemplary embodiment, a control method of an ultrasound apparatus, includes acquiring ultrasound data regarding a subject, acquiring vital data regarding the subject based on the acquired ultrasound data, and determining suitability of injection to a corresponding region based on the acquired vital data.

The vital data regarding the subject may be at least one selected from the group including hardness of the skin, the presence of varicose veins, and the presence of damaged blood vessels.

Determination of injection suitability may include determining that injection is not suitable if hardness of the skin reaches or exceeds a predetermined reference value, if varicose veins are present, or if damaged blood vessels are present.

The control method may further include visibly or audibly outputting the result of determination of injection suitability.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1A is a sectional view showing an internal configuration of a subject;

FIG. 1B is a sectional view showing a position of an injection needle inserted into the subject;

FIGS. 2A to 2C are views showing tools used for insulin injection;

FIG. 3 is a view showing insulin injection candidate regions of the subject;

FIG. 4A is a control block diagram of an ultrasound apparatus according to an exemplary embodiment;

FIG. 4B is a control block diagram showing a detailed configuration of a data acquirer included in the ultrasound apparatus shown in FIG. 4A;

FIG. 5 is a control block diagram of the ultrasound apparatus that further includes a map generator;

FIG. 6 is a view showing one example of a needle length map generated by the map generator;

FIG. 7 is a view showing one example of an applicator that enables self-administration using the needle length map;

FIG. 8A is a control block diagram of an ultrasound apparatus according to another exemplary embodiment;

FIG. 8B is a control block diagram showing a detailed configuration of a data acquirer included in the ultrasound apparatus shown in FIG. 8A;

FIG. 9 is a control block diagram of the ultrasound apparatus that further includes a map generator;

FIG. 10 is a view showing one example of an injection region map generated by the map generator;

FIG. 11 is a control block diagram of an ultrasound apparatus according to another exemplary embodiment;

FIG. 12 is a control block diagram of the ultrasound apparatus that further includes a map generator;

FIG. 13 is a view showing one example of an injection map generated by the map generator;

FIG. 14 is a control block diagram of an ultrasound apparatus for control of a syringe;

FIGS. 15A to 15C are side sectional views of a probe module to which the syringe is coupled;

FIG. 16 is a flowchart showing an ultrasound apparatus control method including selection of needle length;

FIG. 17 is a flowchart showing an ultrasound apparatus control method including generation of a needle length map;

FIG. 18 is a flowchart showing an ultrasound apparatus control method including determination of injection suitability.

FIG. 19 is a flowchart showing an ultrasound apparatus control method including generation of an injection region map;

FIG. 20 is a flowchart showing an ultrasound apparatus control method including determination of injection suitability and selection of needle length;

FIG. 21 is a flowchart showing an ultrasound apparatus control method including generation of an injection map containing data regarding injection suitability as well as data regarding needle length;

FIG. 22 is a flowchart showing an ultrasound apparatus control method including auto-control of needle length; and

FIG. 23 is a flowchart showing an ultrasound apparatus control method including determination of injection suitability and auto-control of needle length.

### DETAILED DESCRIPTION OF EXEMPLARY EMBODIMENTS

Reference will now be made in detail to the exemplary embodiments, examples of which are illustrated in the accompanying drawings, wherein like reference numerals refer to like elements throughout.

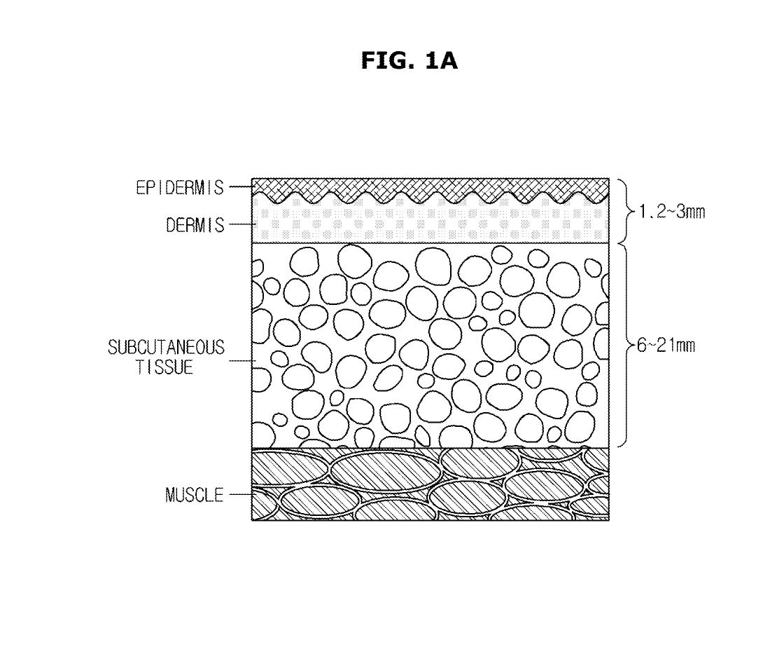

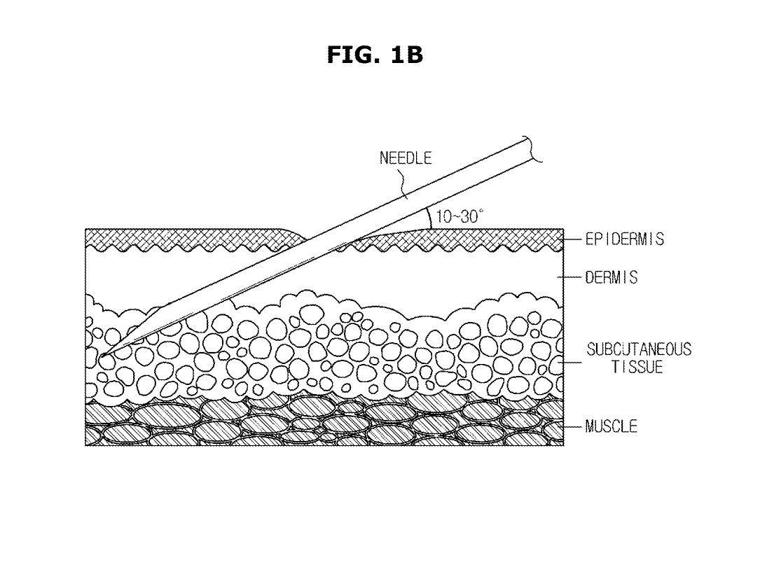

FIG. 1A is a sectional view showing an internal configuration of a subject, and FIG. 1B is a sectional view showing a position of an injection needle inserted into the subject.

Referring to FIG. 1A, the subject, more particularly, a human body consists of the skin including the epidermis and dermis, subcutaneous tissue below the skin, and muscles below the subcutaneous tissue. The subcutaneous tissue is mainly fatty and called subcutaneous fat.

Among drugs administered to patients, drugs administered via injection differ in terms of administration positions, i.e. insertion position of an injection needle. Injection into the skin is called intra-dermal injection, injection into subcutaneous tissue is called subcutaneous injection, and injection into muscles is called intramuscular injection.

For example, the Bacillus Calmette-Guérin (BCG) vaccine is administered via intra-dermal injection, vaccines against hepatitis B, influenza and hepatitis A are administered via intramuscular injection, and vaccines against Japanese encephalitis are administered via subcutaneous injection.

Insulin is administered to diabetic patients who cannot produce insulin on their own, via subcutaneous injection as exemplarily shown in FIG. 1B. Insulin is not administered only once. Instead, insulin is administered to diabetic patients periodically, often for the life of the patient. In consideration of self-administration of diabetic patients, safe and effective injection of insulin is important for diabetes care.

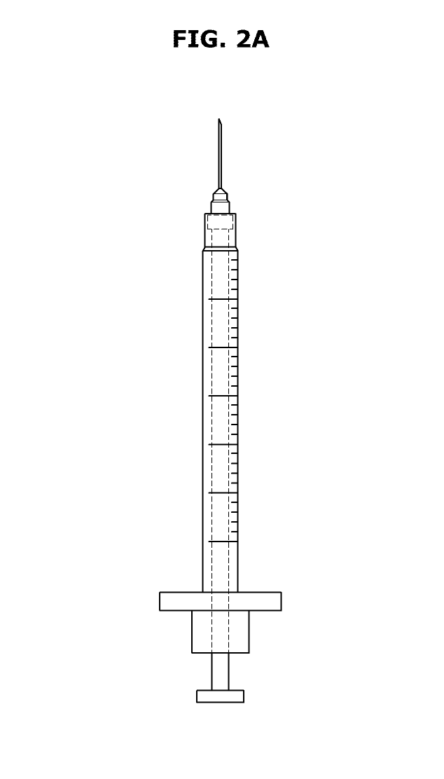

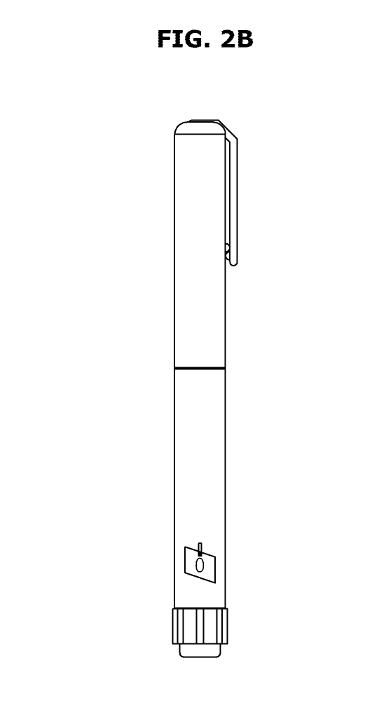

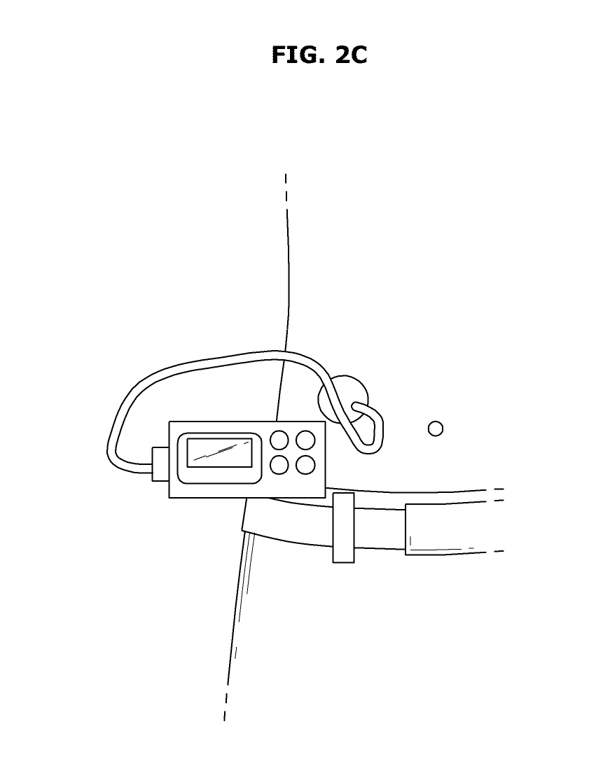

FIGS. 2A to 2C are views showing tools used for insulin injection.

Insulin injection tools include a syringe as exemplarily shown in FIG. 2A, a pen-shaped syringe as exemplarily shown in FIG. 2B, and an insulin pump as exemplarily shown in FIG. 2C.

Explaining operation of insulin injection tools in brief, the syringe shown in FIG. 2A is a standard syringe filled with insulin that is administered into the body via a needle inserted into an injection region of the patient's body as a plunger is pushed. The pen-shaped syringe, e.g., insulin pen, shown in FIG. 2B is configured to administer insulin into the body as a needle thereof protrudes outward when a rear end of the syringe is pushed. The insulin pump shown in FIG. 2C has a shape of a pager and is connected to an injection needle fixedly inserted into subcutaneous tissue through a hose to supply insulin for a preset time or when the patient pushes a button.

Insulin injection tools may be appropriately selected according to patient health factors and various other conditions. For example, if it is necessary to adjust insulin mixing rates or to observe insulin administration with the naked eye, the syringe shown in FIG. 2A may be used. If simplified injection is necessary in consideration of movement of the patient or needle phobia, the pen-shaped syringe shown in FIG. 2B may be used. If several insulin administrations a day are necessary or severe hypoglycemia occurs, the insulin pump shown in FIG. 2C may be used.

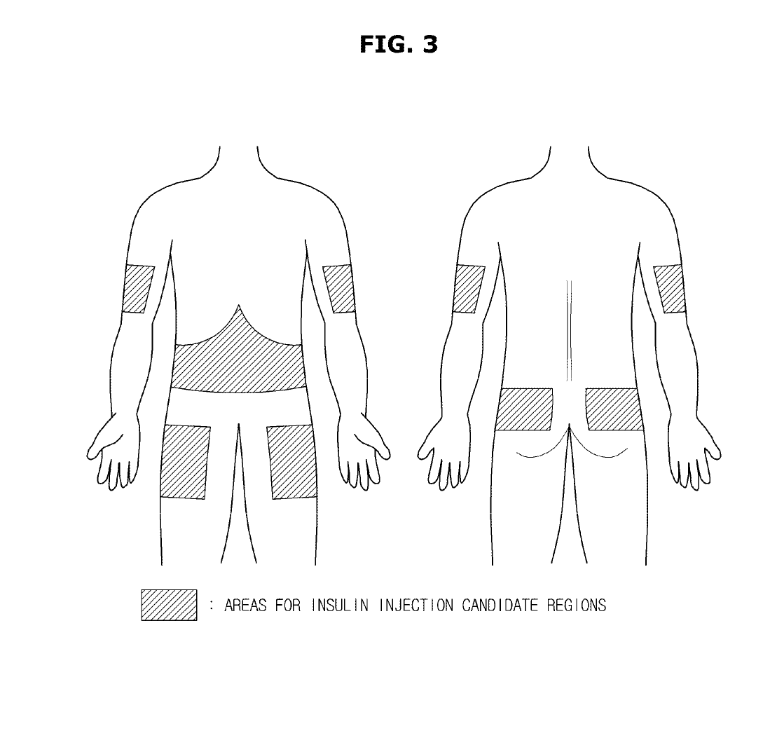

FIG. 3 is a view showing shaded areas including insulin injection candidate regions, i.e., candidate regions for insulin injection, of the subject.

Referring to FIG. 3, insulin may be administered to specific regions, such as the abdomen, arm, thigh, hip, and the like. These regions differ in terms of the thickness of subcutaneous tissue, and thus selection of appropriate needle length per injection region is necessary. However, since the thickness of subcutaneous tissue per injection region differs according to physical characteristics, such as gender, age, build, and the like, selecting an injection needle suitable for each insulin injection region may be difficult.

Accordingly, the ultrasound apparatus according to an exemplary embodiment may measure the thickness of subcutaneous tissue using an ultrasound probe to accurately select an injection needle suitable for a corresponding insulin injection region.

All of the insulin injection tools as explained with reference to FIGS. 2A to 2C include a needle to be inserted into the subject. Therefore, the ultrasound apparatus according to an exemplary embodiment may be applied to any one of the insulin injection tools. That is, regardless of whether the standard syringe, the pen-shaped syringe, or the insulin pump is used, the ultrasound apparatus according to an exemplary embodiment may achieve selection of an appropriate injection needle. Hereinafter, operation of the ultrasound apparatus according to an exemplary embodiment will be described.

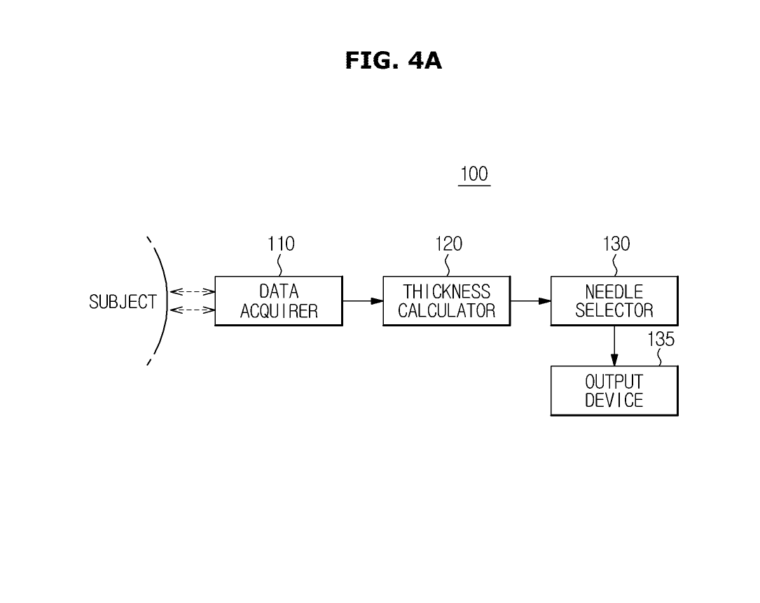

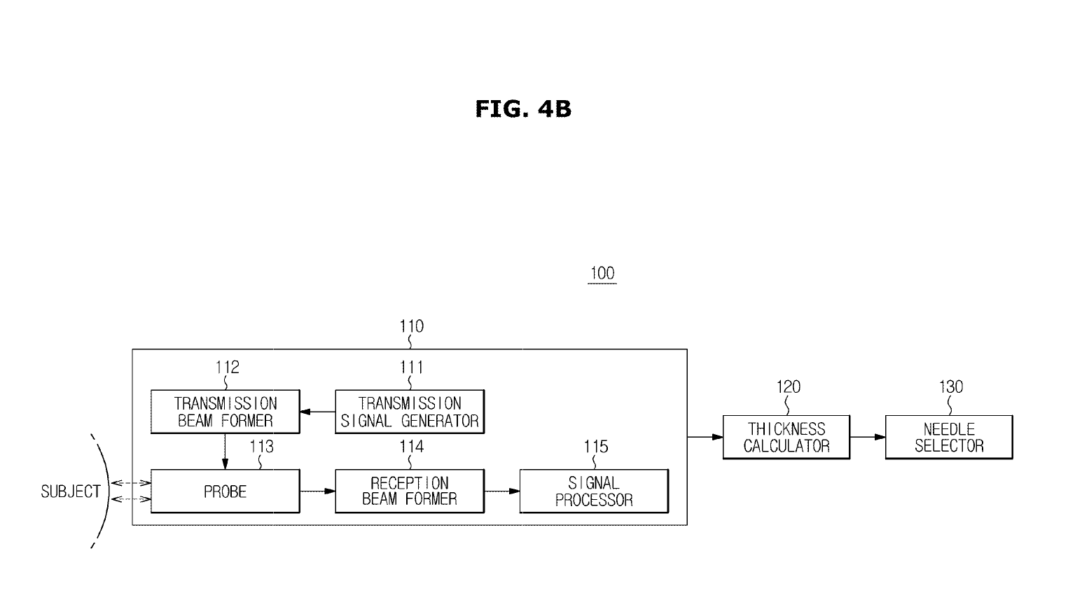

FIG. 4A is a control block diagram of an ultrasound apparatus according to an exemplary embodiment, and FIG. 4B is a control block diagram showing a detailed configuration of a data acquirer included in the ultrasound apparatus shown in FIG. 4A.

Referring to FIG. 4A, the ultrasound apparatus, designated by reference numeral 100, includes a data acquirer 110 to acquire ultrasound data regarding the subject, a thickness calculator 120 to calculate the thickness of subcutaneous tissue based on the ultrasound data regarding the subject, and a needle selector 130 to select a needle corresponding to the calculated thickness of subcutaneous tissue. In the present exemplary embodiment, the subject may be a human body, and more particularly an insulin-dependent diabetic patient. In an exemplary embodiment, each of the data acquirer 110, thickness calculator 120, and the needle selector 130 may be implemented in hardware or software.

The data acquirer 110 acquires the ultrasound data regarding the subject by transmitting ultrasound to the subject and receiving the ultrasound reflected from the interior of the subject.

The data acquirer 110, as exemplarily shown in FIG. 4B, includes a transmission signal generator 111 to generate signals to be transmitted to the subject, a transmission beam former 112 to focus transmission signals, a probe 113 to transmit and receive ultrasound, a reception beam former 114 to focus reception signals, and a signal processor 115 to perform signal processing on the reception signals. Each of the aforementioned elements of the data acquirer may be implemented in hardware or software.

The probe 113 includes a plurality of transducer elements for conversion between ultrasound and electric signals. The transmission beam former 112 appropriately delays input of pulses to the respective transducer elements to focus ultrasound beams.

The transducer elements convert the focused transmission signals into ultrasound to transmit the ultrasound to the subject. In addition, the transducer elements receive ultrasound echo reflected from the subject to convert the ultrasound echo into electric signals. The electric signals are reception signals.

Ultrasound echo is input at different times to the respective transducer elements. The reception beam former 114 converts analog reception signals into digital signals, and focuses the digital reception signals in consideration of positions and focal points of the transducer elements.

The signal processor 115 generates ultrasound data required for thickness calculation via signal processing, such as band-pass filtering, gain control, and the like.

The thickness calculator 120 calculates the thickness of subcutaneous tissue based on the ultrasound data regarding the subject. Ultrasound data includes data regarding the interior tissue structure of the subject. In one example, materials differently reflect ultrasound, and therefore generate different magnitudes of ultrasound data. Accordingly, the boundary between different materials may be detected when the magnitude of ultrasound data varies or when the waveform of ultrasound data exceeds a critical value, whereby the thickness of a specific material may be calculated. The thickness calculator 120 may calculate the thickness of subcutaneous tissue in the above-described manner, and may also calculate the thickness of skin composed of the epidermis and dermis as necessary.

The needle selector 130 selects a needle corresponding to the calculated thickness of subcutaneous tissue. The selected needle has a length corresponding to the calculated thickness of subcutaneous tissue. The needle length corresponding to the thickness of subcutaneous tissue may differ according to injection methods.

If injection is performed in an upright state of a needle, i.e., the needle is inserted at an angle of approximately 90 degrees, a needle, the length of which is greater than the thickness of skin and less than the sum of the thickness of skin and the thickness of subcutaneous tissue, may be selected as a needle corresponding to the thickness of subcutaneous tissue. If injection is performed in a tilted state of a needle, i.e., the needle is inserted at an angle less than 90 degrees, e.g., 45 degrees or less, between 10 and 30 degrees, between other angles, a needle, the length of which ensures insertion of a needle tip into subcutaneous tissue in consideration of the tilting angle, may be selected. In addition, if injection is performed in a state in which the skin is pulled away from the body, e.g., the skin is grasped between the fingers or the skin is pinched, a needle, the length of which ensures insertion of a needle tip into subcutaneous tissue in consideration of an increased thickness of subcutaneous tissue, may be selected.

The needle selector 130 may select a needle based on the method of injection, may select only a needle suitable for an injection method selected by a user, or may select a needle based on injection using an upright needle in a state in which the skin is not pulled away from the body.

The needle selector 130 may select a needle based on a needle length calculation algorithm stored therein, and may search for and select a needle corresponding to the calculated thickness of subcutaneous tissue using a needle database.

Meanwhile, the thickness of a needle may be represented by gauge (G) and differs according to needle length. The needle thickness is in a range of 14 G to 32 G. As the gauge increases, the needle thickness, i.e. the needle diameter decreases as well as the needle length. Thus, selection of a needle by the needle selector 130 includes selection of needle thickness as well as selection of needle length.

The ultrasound apparatus 100 may include an output device 135 (FIG. 4A) which may be a display device, a speaker or both. The display device visibly outputs the result of selection by the needle selector 130 to the user. The speaker may audibly output the result of selection by the needle selector 130 to the user.

The ultrasound apparatus 100 may be owned by the patient for insulin self-administration, or may be used in a hospital. That is, the user of the ultrasound apparatus 100 according to an exemplary embodiment may be a medical technician, patient, or patient's caretaker.

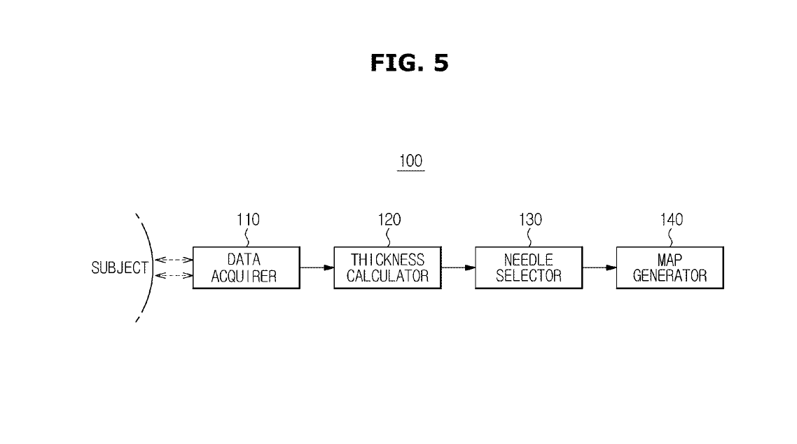

FIG. 5 is a control block diagram of the ultrasound apparatus 100 that further includes a map generator.

Referring to FIG. 5, the ultrasound apparatus 100 may further include a map generator 140 to generate a needle length map, in addition to the above-described components. In an exemplary embodiment, the map generator 140 may be implemented in hardware or software.

The map generator 140 generates a needle length map by matching a selected needle length for a particular body region of the subject, to a corresponding region. To this end, the ultrasound apparatus 100 acquires ultrasound data regarding a plurality of injection candidate regions in which insulin injection will be performed to calculate the thickness of subcutaneous tissue, and selects a needle length corresponding to the calculated thickness of subcutaneous tissue. As exemplarily shown in FIG. 3, insulin may be injected into the abdomen, arm, thigh, hip, and the like. The ultrasound apparatus 100 may perform acquisition of ultrasound data regarding the aforementioned regions, thickness calculation of subcutaneous tissue, and selection of needle length.

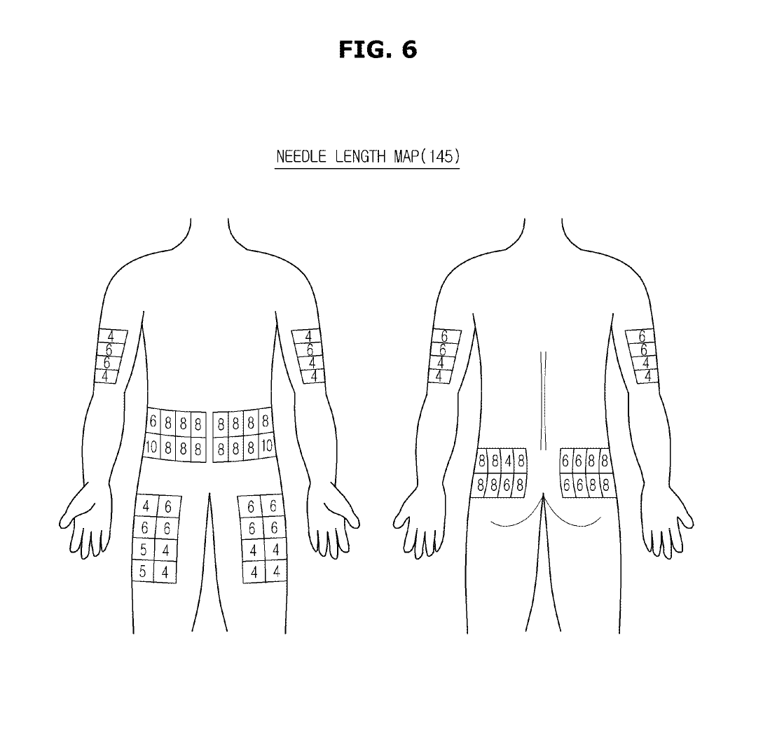

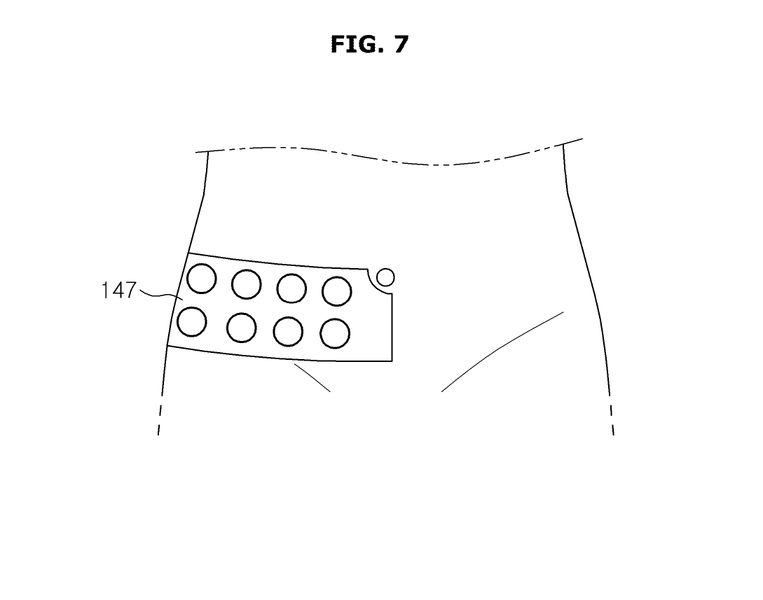

FIG. 6 is a view showing one example of the needle length map generated by the map generator, and FIG. 7 is a view showing one example of an applicator that enables self-administration using the needle length map.

In one example, the map generator 140 may generate a needle length map 145 as exemplarily shown in FIG. 6. The body of the subject shown in FIG. 6 may be a real photograph of the subject, or may be an avatar or a representative image corresponding to the subject. In the former case, the ultrasound apparatus 100 may include a camera to capture an image of the subject. As such, to generate the needle length map 145, the ultrasound apparatus 100 may capture an image of the subject using the camera, and mark a selected needle length for each insulin injection candidate region on the captured image of the subject. The unit of needle length marked on the map 145 of FIG. 6 may be mm.

In the latter case, the needle length may be marked on an avatar or a representative image having the shape of a human being. The avatar or the representative image may be equally applied to all subjects, or may have a shape suitable for physical conditions of a real subject, i.e. may have different shapes on a per subject basis. If the avatar or the representative image has physical conditions of a real subject, insulin injection candidate regions of the subject may be more accurately understood from the needle length map 145.

The needle length map 145 generated by the map generator 140 may be provided to the patient in the form of a file or other output formats. For example, the needle length map 145 may be provided in the form of a file or printed output to allow the patient to view the needle length map 145 via an appliance, such as a PC, smart-phone, or the like. The map 145 may be provided in an appropriate form according to, e.g., the preference and age of the patient. The ultrasound apparatus 100 according to an exemplary embodiment is not limited as to data provision methods so long as transmission of data contained in the map 145 is ensured.

The patient may perform insulin self-administration while viewing the needle length map 145. To this end, an applicator 147 as exemplarily shown in FIG. 7 may be provided. Apertures of the applicator 147 represent injection candidate regions. The applicator 147 is attached about the navel or is placed on the skin by using the navel as a point of reference or other parts of the body as a point of reference, and insulin is injected into the injection candidate regions marked via the apertures.

Referring to the needle length map 145 of FIG. 6, on the basis of a first line of the applicator located at the left side of the navel, a first injection candidate region from the left may be subjected to insulin injection using a needle having a length of 6 mm and a fourth injection region from the left may be subjected to insulin injection using a needle having a length of 8 mm.

The applicator 147 may allow the patient to inject insulin using a needle, the length of which corresponds to a specific injection candidate region, and may prevent the patient from repeatedly injecting insulin in the same region.

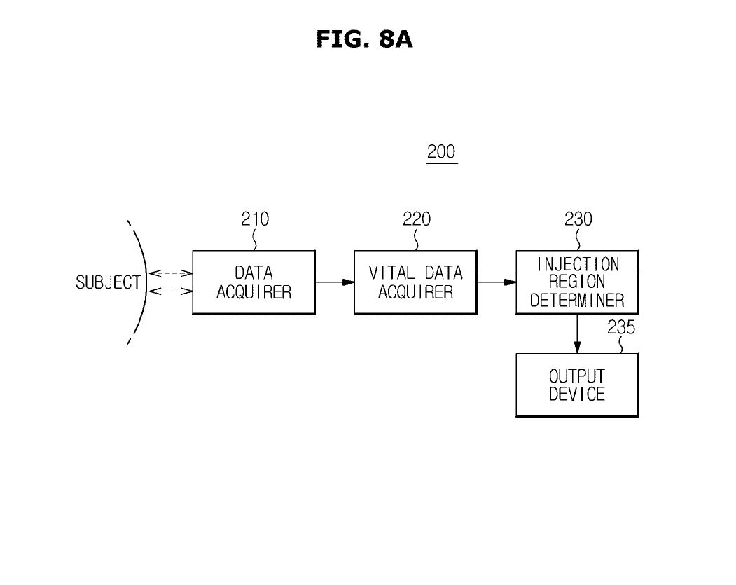

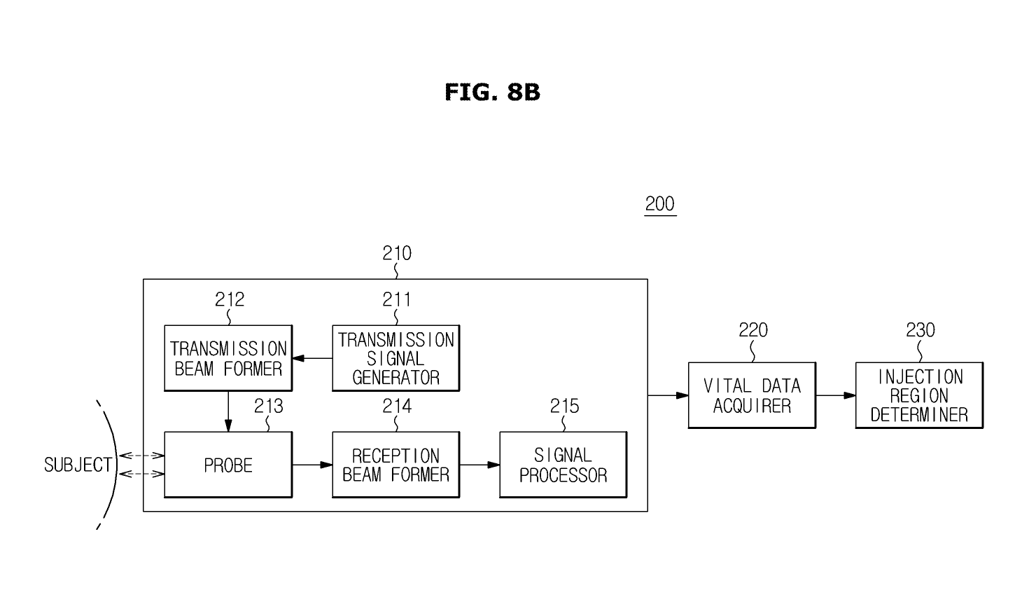

FIG. 8A is a control block diagram of an ultrasound apparatus according to another exemplary embodiment, and FIG. 8B is a control block diagram showing a detailed configuration of a data acquirer included in the ultrasound apparatus shown in FIG. 8A.

Referring to FIG. 8A, the ultrasound apparatus, designated by reference numeral 200, according to another exemplary embodiment includes a data acquirer 210 to acquire ultrasound data regarding the subject, a vital data acquirer 220 to acquire vital data regarding the subject based on the ultrasound data, and an injection region determiner 230 to determine suitability of injection to a corresponding region based on the vital data regarding the subject. In an exemplary embodiment, each of the data acquirer 210, vital data acquirer 220, and the injection region determiner 230 may be implemented in hardware or software.

Injection is not preferable with regard to body regions having excessively hard skin, varicose veins, damaged blood vessels, or inflamed tissue as well as burned or scarred regions. The burned or scarred regions are visually distinguishable and accidental insulin injection to these regions may be prevented, whereas the other affected regions may be difficult to visually identify.

Accordingly, if the data acquirer 210 acquires ultrasound data regarding a specific region of the subject, the vital data acquirer 220 acquires at least one vital data selected from the group including hardness of the skin, the presence of damaged blood vessels, the presence of inflamed tissue, the presence of varicose veins, and the like in the corresponding region based on the acquired ultrasound data.

Referring to FIG. 8B, the data acquirer 210 includes a transmission signal generator 211 to generate signals to be transmitted to the subject, a transmission beam former 212 to focus transmission signals, a probe 213 to transmit and receive ultrasound, a reception beam former 214 to focus reception signals, and a signal processor 215 to perform signal processing on the reception signals. In an exemplary embodiment, each of the aforementioned elements of the data acquirer 210 may be implemented in hardware or software.

The probe 213 includes a plurality of transducer elements for conversion between ultrasound and electric signals. The transmission beam former 212 appropriately delays input of pulses to the respective transducer elements to focus ultrasound beams.

The transducer elements convert the focused transmission signals into ultrasound to transmit the ultrasound to the subject. In addition, the transducer elements receive ultrasound echo reflected from the subject to convert the ultrasound echo into electric signals. The electric signals are reception signals.

Ultrasound echo is input at different times to the respective transducer elements. The reception beam former 214 converts analog reception signals into digital signals, and focuses the digital reception signals in consideration of positions and focal points of the transducer elements.

The signal processor 215 generates ultrasound data required for acquisition of vital data via signal processing, such as band-pass filtering, signal amplification, gain control, and the like.

Referring again to FIG. 8A, the vital data acquirer 220 acquires vital data regarding the subject based on the ultrasound data acquired by the data acquirer 210. As described above, in the present exemplary embodiment, the vital data regarding the subject may be at least one vital data selected from the group including hardness of the skin, the presence of damaged blood vessels, the presence of inflamed tissue, and the presence of varicose veins. The vital data is used to determine suitability of insulin injection to a corresponding region. In addition to the aforementioned data, the vital data acquired by the vital data acquirer 220 may include other data so long as such data aids in determination of injection candidate regions.

In one example, to acquire hardness of the skin, ultrasound elasticity imaging may be adopted. More specifically, the data acquirer 210 transmits ultrasound in a state in which stress is not applied to the subject to receive ultrasound echo, and then transmits ultrasound in a state in which stress is applied to the subject to receive ultrasound echo, thereby acquiring ultrasound data. The vital data acquirer 220 calculates the strain rate of a corresponding region using the ultrasound data with regard to the state in which stress is applied and the state in which stress is not applied. A lower strain rate means greater hardness. Thus, the calculated strain rate is vital data indicating hardness of a corresponding region. In addition, the strain rate may be used to determine whether or not inflamed tissue is present in a corresponding region.

The presence of damaged blood vessels and the presence of varicose veins may be determined using ultrasound Doppler techniques. Doppler techniques enable real-time detection of blood flow in a corresponding region. More specifically, color Doppler techniques enable detection of directionality of blood flow, and power Doppler techniques enable detection of continuity of blood flow. In addition, pulse-Doppler techniques enable acquisition of quantitative data, such as the flow rate of blood. Accordingly, the vital data acquirer 220 may determine whether or not varicose veins are present via color Doppler techniques, and may detect damaged blood vessels via power Doppler techniques or pulse-Doppler techniques.

The injection region determiner 230 determines suitability of insulin injection to a corresponding region based on the vital data acquired by the vital data acquirer 220. In one example, if hardness of a corresponding region reaches or exceeds a reference value, the corresponding region may be determined as an insulin-injection prohibition region. In addition, if damaged blood vessels, inflamed tissue or varicose veins are present in a corresponding region, the corresponding region may likewise be determined as an insulin-injection prohibition region.

The ultrasound apparatus 200 may include an output device 235 such as a display device, a speaker, or both. The display device visibly outputs the result of determination by the injection region determiner 230 to the user. The speaker may audibly output the result of determination by the injection region determiner 230 to the user.

The ultrasound apparatus 200 may be owned by the patient for insulin self-administration, or may be used in a hospital. That is, the user of the ultrasound apparatus 200 according to the present exemplary embodiment may be a medical technician, patient, or patient's caretaker.

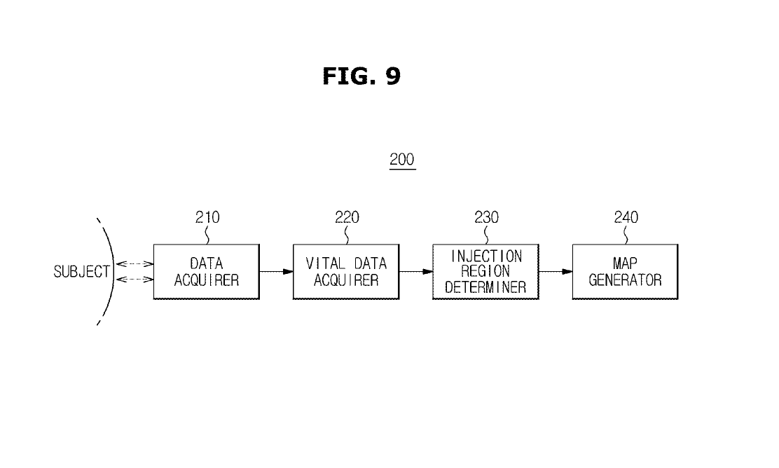

FIG. 9 is a control block diagram of the ultrasound apparatus 200 that further includes a map generator.

Referring to FIG. 9, the ultrasound apparatus 200 may further include a map generator 240 to generate an injection region map, in addition to the above-described components (including or not including the output device 235). The map generator 240 may be implemented in hardware or software.

The map generator 240 generates an injection region map by matching suitability of injection to each body region of the subject. To this end, the ultrasound apparatus 200 acquires ultrasound data regarding a plurality of injection candidate regions in which insulin injection will be performed to acquire vital data, and determines suitability of injection. As exemplarily shown in FIG. 3, insulin may be injected into the abdomen, arm, thigh, hip, and the like. The ultrasound apparatus 200 may perform acquisition of ultrasound data regarding the aforementioned regions, acquisition of vital data, and determination of injection suitability.

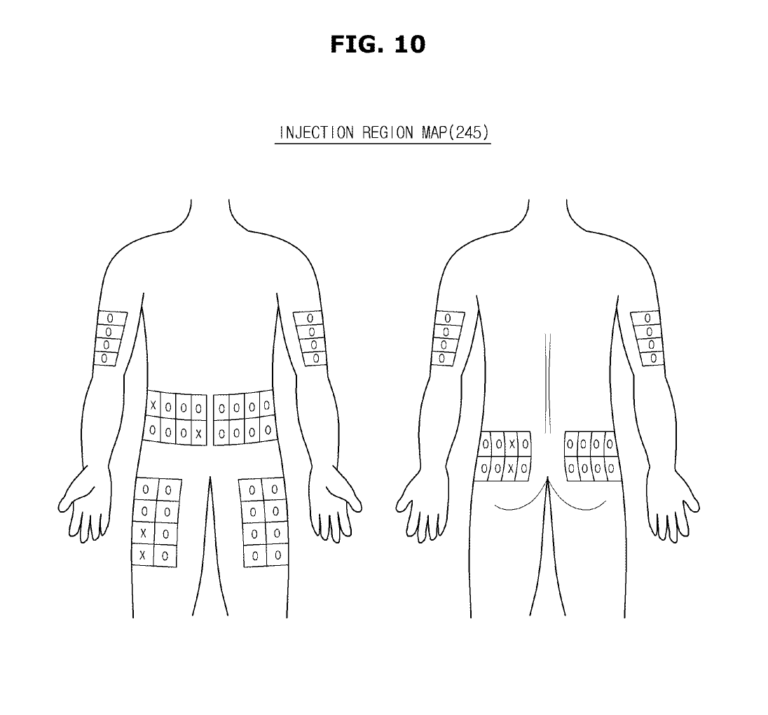

FIG. 10 is a view showing one example of the injection region map generated by the map generator.

The map generator 240 may generate an injection region map 245 as exemplarily shown in FIG. 10. The body of the subject shown in FIG. 10 may be a real photograph of the subject, or may be an avatar or a representative image corresponding to the subject. In the former case, the ultrasound apparatus 200 may include a camera to capture an image of the subject. As such, to generate the injection region map 245, the ultrasound apparatus 200 may capture an image of the subject using the camera, and mark injection suitability for each insulin injection candidate region on the captured image of the subject.

In the latter case, injection suitability may be marked on an avatar or a representative image having the shape of a human being. The avatar or the representative image may be equally applied to all subjects, or may have a shape suitable for physical conditions of a real subject, i.e. may have different shapes on a per subject basis. If the avatar or the representative image has physical conditions of a real subject, insulin injection candidate regions of the subject may be more accurately understood from the injection region map 245.

The injection region map 245 generated by the map generator 240 may be provided to the patient in the form of a file or other output formats. For example, the needle length map 145 may be provided in the form of a file or printed output to allow the patient to view the injection region map 245 via an appliance, such as a PC, smart-phone, or the like. The injection region map 245 may be provided in an appropriate form according to, e.g., the preference and age of the patient. The ultrasound apparatus 200 according to the present exemplary embodiment is not limited as to data provision methods so long as transmission of data contained in the injection region map 245 is ensured.

The patient may perform insulin self-administration while viewing the injection region map 245. To this end, the applicator 147 as exemplarily shown in FIG. 7 may be provided. The applicator 147 is attached about the navel, and insulin is injected into the injection candidate regions marked via the apertures.

Referring to the injection region map 245 of FIG. 10, on the basis of a first line of the applicator located at the left side of the navel, a first injection region from the left as well as other regions where injection prohibition marks are provided are unsuitable for insulin injection. Injection candidate regions and injection prohibition regions may be indicated respectively by ◯ and X as exemplarily shown in FIG. 10. Other marks may be used so long as the user can identify the injection candidate regions and injection prohibition regions.

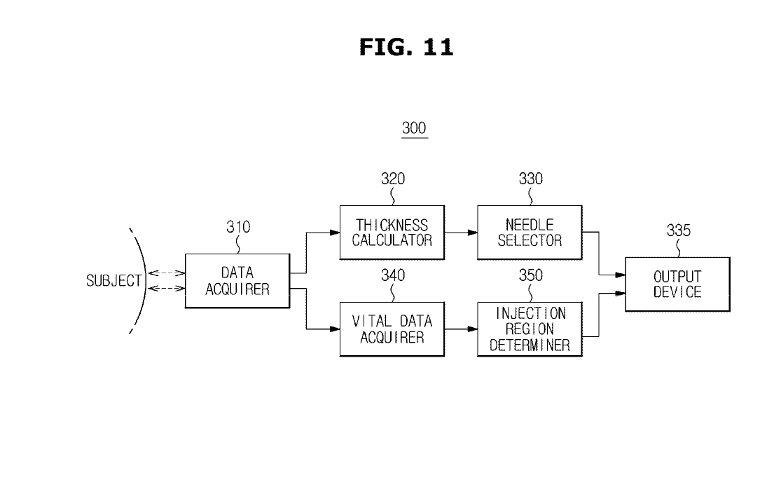

FIG. 11 is a control block diagram of an ultrasound apparatus according to another exemplary embodiment.

In the above-described exemplary embodiments, the ultrasound apparatus 100 of FIG. 4A is configured to select a needle length corresponding to the calculated thickness of subcutaneous tissue of an injection candidate region, and the ultrasound apparatus 200 of FIG. 8A is configured to determine suitability of injection to a corresponding region based on acquired vital data. The ultrasound apparatus, designated by reference numeral 300, according to the present exemplary embodiment may determine suitability of injection to a corresponding injection candidate region, and select a needle length corresponding to the injection candidate region if it is determined that the corresponding region is suitable for injection, thereby ensuring safe and effective insulin injection.

To this end, the ultrasound apparatus 300 includes a data acquirer 310 to acquire ultrasound data regarding the subject, a thickness calculator 320 to calculate the thickness of subcutaneous tissue based on the ultrasound data regarding the subject, a needle selector 330 to select a needle corresponding to the calculated thickness of subcutaneous tissue, a vital data acquirer 340 to acquire vital data regarding the subject based on the ultrasound data regarding the subject, and an injection region determiner 350 to determine suitability of injection to a corresponding region based on the vital data regarding the subject. Each of the aforementioned elements of the ultrasound apparatus may be implemented in hardware or software.

Operations of the respective components are equal to those of the above-described exemplary embodiments, and thus a detailed description thereof will be omitted hereinafter.

If the data acquirer 310 acquires ultrasound data regarding the subject, the vital data acquirer 340 acquires vital data regarding the subject based on the acquired ultrasound data, and the injection region determiner 350 determines suitability of injection to a corresponding region based on the acquired vital data. Then, the thickness calculator 320 calculates the thickness of subcutaneous tissue based on the acquired ultrasound data, and the needle selector 330 selects a needle length corresponding to the thickness of subcutaneous tissue.

Determination of suitability of injection to the corresponding region and selection of needle length may be performed independently of each other and the results may be provided to the user via the output device 335 which may be the display device or the speaker, or both. In an exemplary embodiment, the output device 335 has two parts so that one part corresponds to the needle selector 330 and another part corresponds to the injection region determiner 350. Calculation of the thickness of subcutaneous tissue and selection of needle length may be performed only when injection suitability is determined, such that the selected needle length is provided to the user via the display device or the speaker.

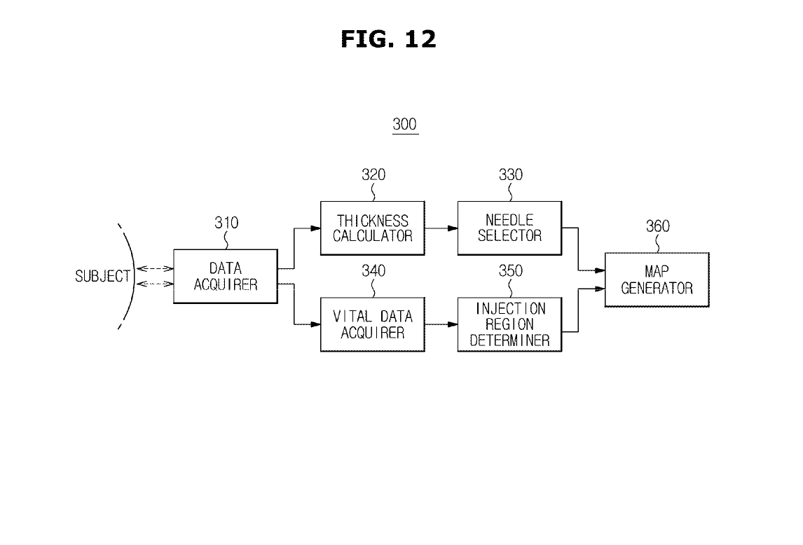

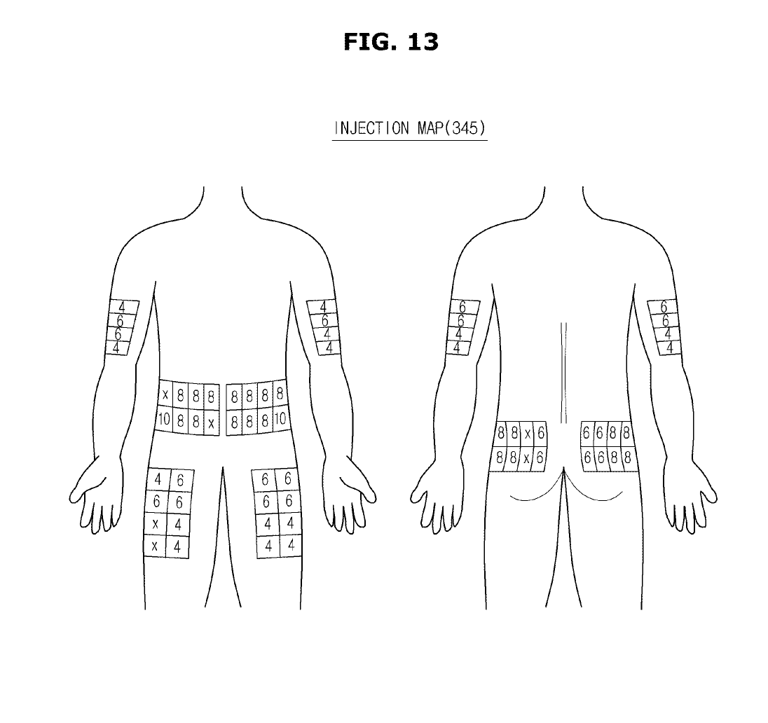

FIG. 12 is a control block diagram of the ultrasound apparatus 300 that further includes a map generator, and FIG. 13 is a view showing one example of an injection map generated by the map generator.

Referring to FIG. 12, in the same manner as in the above-described exemplary embodiments, the ultrasound apparatus 300 according to the present exemplary embodiment further includes a map generator 360 to generate an injection map on which suitability of injection to an injection candidate region and a corresponding needle length are marked. In an exemplary embodiment, the map generator 360 may be implemented in hardware or software.

To this end, the ultrasound apparatus 300 acquires ultrasound data regarding a plurality of regions in which insulin injection will be performed, and performs determination of injection suitability as well as selection of needle length. As exemplarily shown in FIG. 3, insulin may be injected to the arm, abdomen, thigh, hip, and the like, and the ultrasound apparatus 300 may perform determination of injection suitability as well as selection of needle length with regard to the aforementioned regions.

In one example, the map generator 360 may generate an injection map 345 as exemplarily shown in FIG. 13.

The body of the subject shown in FIG. 13 may be a real photograph of the subject, or may be an avatar or a representative image corresponding to the subject. In the former case, the ultrasound apparatus 300 may include a camera to capture an image of the subject. As such, to generate the injection map 345, the ultrasound apparatus 300 may capture an image of the subject using the camera, and mark a selected needle length for each insulin injection candidate region on the captured image of the subject. Data of the needle length map 145 as well as data of the injection region map 245 are marked on the injection map 345. That is, injection prohibition marks are provided in injection prohibition regions, and needle length corresponding to each injection candidate region is marked in the injection candidate region. The unit of needle length marked on the map 345 of FIG. 13 may be mm.

In the latter case, the needle length may be marked on an avatar or a representative image having the shape of a human being. The avatar or the representative image may be equally applied to all subjects, or may have a shape suitable for physical conditions of a real subject, i.e. may have different shapes on a per subject basis. If the avatar or the representative image has physical conditions of a real subject, insulin injection candidate regions of the subject may be more accurately understood from the injection map 345.

The injection map 345 generated by the map generator 340 may be provided to the patient in the form of a file or other output formats. For example, the injection map 345 may be provided in the form of a file or printed output to allow the patient to view the injection map 345 via an appliance, such as a PC, smart-phone, or the like. The injection map 345 may be provided in an appropriate form according to, e.g., the preference and age of the patient. The ultrasound apparatus 300 according to the present exemplary embodiment is not limited as to data provision methods so long as transmission of data contained in the injection map 345 is ensured.

The patient may perform insulin self-administration while viewing the injection map 345. To this end, the applicator 147 as exemplarily shown in FIG. 7 may be provided. Injection may not be performed in regions provided with injection prohibition marks, and only injection suitable regions may be subjected to injection using needles having marked lengths.

Although the ultrasound apparatus 100 according to the above described exemplary embodiment functions to select a needle length depending on the thickness of subcutaneous tissue of the subject and provide the user with the selected needle length, an ultrasound apparatus according to another exemplary embodiment may automatically control a needle of a syringe. This will be described hereinafter in detail.

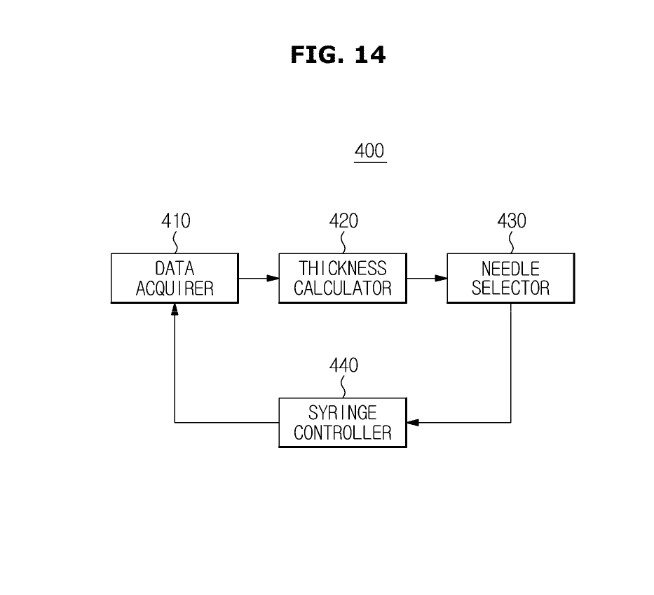

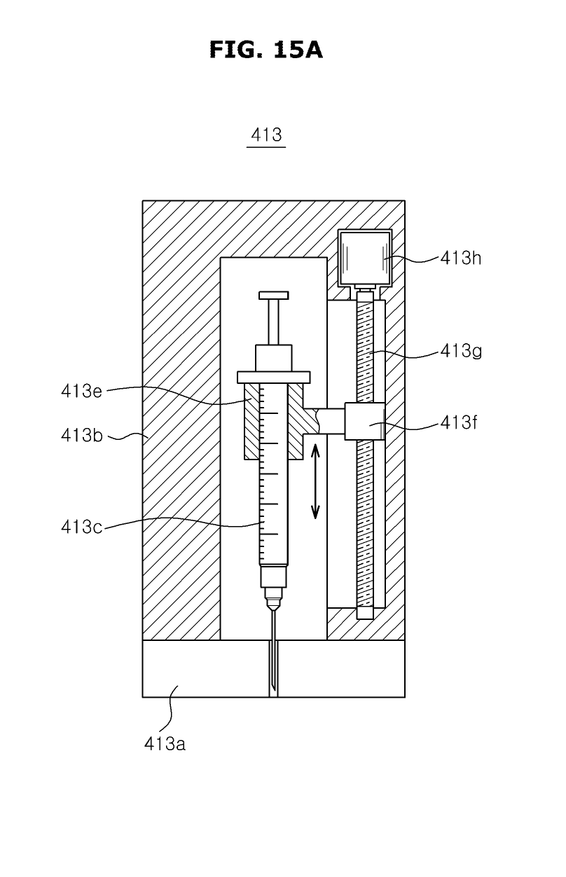

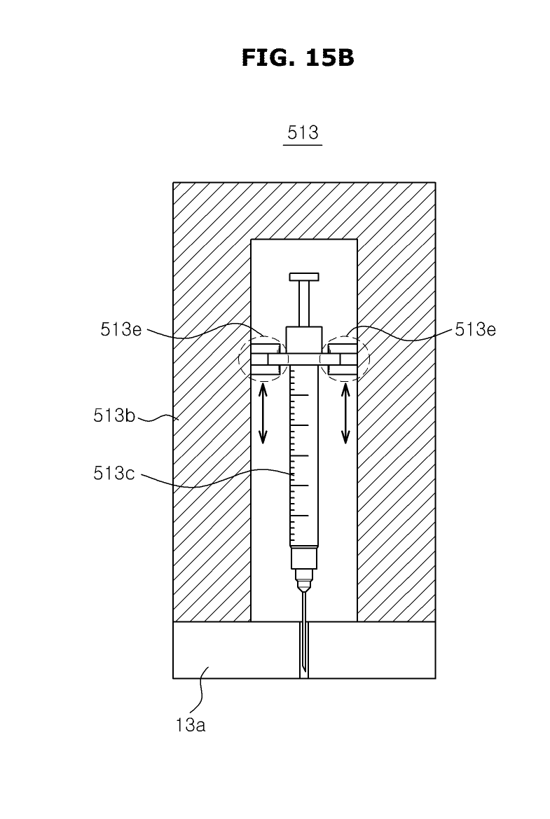

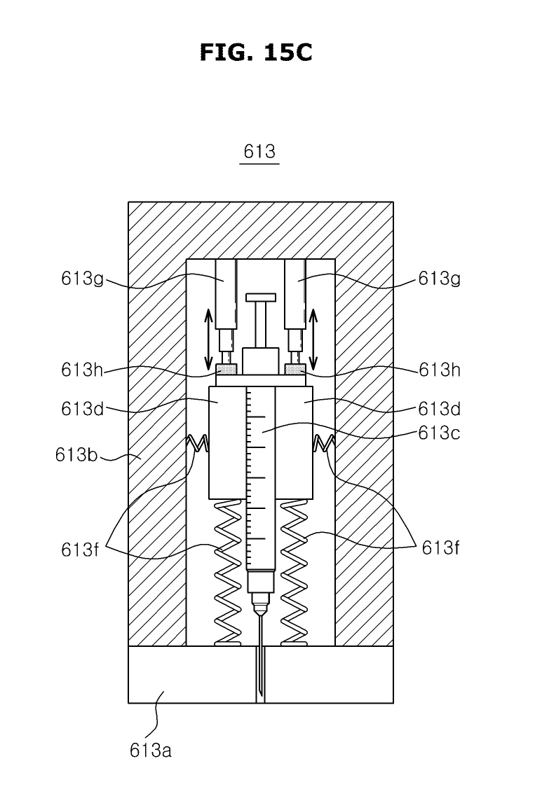

FIG. 14 is a control block diagram of the ultrasound apparatus for control of a syringe, and FIGS. 15A to 15C are side sectional views of a probe module to which the syringe is coupled.

Referring to FIG. 14, the ultrasound apparatus 400 includes a data acquirer 410 to acquire ultrasound data regarding the subject, a thickness calculator 420 to calculate the thickness of subcutaneous tissue based on the ultrasound data regarding the subject, a needle selector 430 to select a needle corresponding to the calculated thickness of subcutaneous tissue, and a syringe controller 440 to control a needle of a syringe according to the selected needle length. Each of the aforementioned elements of the ultrasound apparatus 400 may be implemented in hardware or software.

Operations of the data acquirer 410, the thickness calculator 420, and the needle selector 430 are equal to those of the above-described exemplary embodiment of FIGS. 4A and 4B, and thus a detailed description thereof will be omitted hereinafter.

A probe module included in the data acquirer 410 may include a syringe as exemplarily shown in FIGS. 15A to 15C to perform measurement of the thickness of subcutaneous tissue as well as insulin injection. To this end, the probe module may be configured to automatically move the syringe. The syringe controller 440 calculates an insertion depth of a needle of the syringe mounted in the probe module based on the needle length selected by the needle selector 430 to transmit to the calculated insertion depth to the probe module.

Referring to FIG. 15A, a syringe 413c may be mounted in a probe module 413 and coupled to a probe 413a. A needle of the syringe 413c may be inserted into the subject through a hole of the probe 413a.

The syringe 413c is fixed by a holder 413e surrounding the syringe 413c. In an exemplary embodiment, the holder 413e holds or clamps the barrel of the syringe 413c. An insertion depth of the needle of the syringe 413c is controlled via upward or downward movement of the holder 413e. In one example, as exemplarily shown in FIG. 15A, if the holder 413e is connected to a nut 413f coupled to a screw 413g inside a housing 413b and a motor 413h transmits power to the screw 413g, the holder 413e connected to the nut 413f may be moved upward or downward. The motor 413h may be controlled by the syringe controller 440. The syringe controller 440 controls the driving amount of the motor 413h to allow the needle of the syringe 413c to be inserted to the needle length selected by the needle selector 430. That is, a control signal corresponding to the driving amount is transmitted to the motor 413h.

For example, if the needle length selected by the needle selector 430 is 5 mm, driving of the motor 413h may be controlled to move the needle of the syringe 413c from an initial position of the syringe 413c so as to protrude outward from the probe 413a by a length of 5 mm. The driving amount of the motor 413h corresponding to the selected needle length may be stored in a database, or may be calculated by prestored algorithms.

FIG. 15B is a side sectional view showing another exemplary embodiment of the probe module.

Referring to FIG. 15B, the probe module, designated by reference numeral 513, according to another exemplary embodiment may include a holder 513e configured to hold both upper and lower ends of a syringe 513c. In an exemplary embodiment, the holder 513e is configured to hold a flange of the syringe 513e. The holder 513e may be moved upward or downward like the holder 413e shown in FIG. 15A. To move the holder 413e, a motor, a screw, and a nut as exemplarily shown in FIG. 15A may be provided in a housing 513b. In addition, operation of the syringe controller 440 to insert a needle of the syringe 513c such that the needle has a selected length is equal to the above description.

FIG. 15C is a side sectional view showing a further exemplary embodiment of the probe module.

Referring to FIG. 15C, the probe module, designated by reference numeral 613, according to the present exemplary embodiment includes support members 613d provided at both sides of a syringe 613c and press members 613h to press the support members 613d and the syringe 613c. In an exemplary embodiment, the support members 613d and the press members 613h grip the flange of the syringe 613c and the support members 613d may further grip, hold or clamp the barrel portion of the syringe 613c. The press members 613h are mounted to shafts 613g connected to the top of a housing 613b. The shafts 613g, as exemplarily shown in FIG. 15C, may have a telescopic configuration to move the press members 613h upward or downward, or may be configured so as to be inserted into the housing 613b. Elastic members 613f, such as springs, may be provided below the support members 613d to assist upward or downward movement of the syringe 613c. Additional elastic members 613f may be provided between the support members 613d and an inner wall of the housing 613b to assist installation of different sizes of the syringe 613c. In the present exemplary embodiment, the press members 613h may be moved upon receiving power from the motor, and the driving amount of the motor may be controlled by the syringe controller 440 as described above.

However, the configuration of the probe module 413, 513 or 613 shown in FIGS. 15A to 15C may be adopted in the ultrasound apparatus 400 by way of example, and the exemplary embodiments are not limited thereto.

Although not shown in the drawings, a plunger of the syringe 413c, 513c or 613c may be automatically pushed within the probe module 413, 513 or 613. To this end, a configuration similar to that for movement of the syringe 413c, 513c or 613c may be adopted, and the syringe controller 440 may control driving amount required to push the plunger for adjustment of drug injection amount.

Alternatively, the plunger of the syringe 413c, 513c or 613c may be exposed outwardly to assist the user in directly performing drug injection.

In addition, the ultrasound apparatus 400 may further include the vital data acquirer and the injection region determiner as described above in FIG. 11 to determine suitability of injection to a corresponding region and to control the needle of the syringe if injection is suitable.

Hereinafter, exemplary embodiments with regard to a control method of the ultrasound apparatus according to one aspect of the exemplary embodiment will be described.

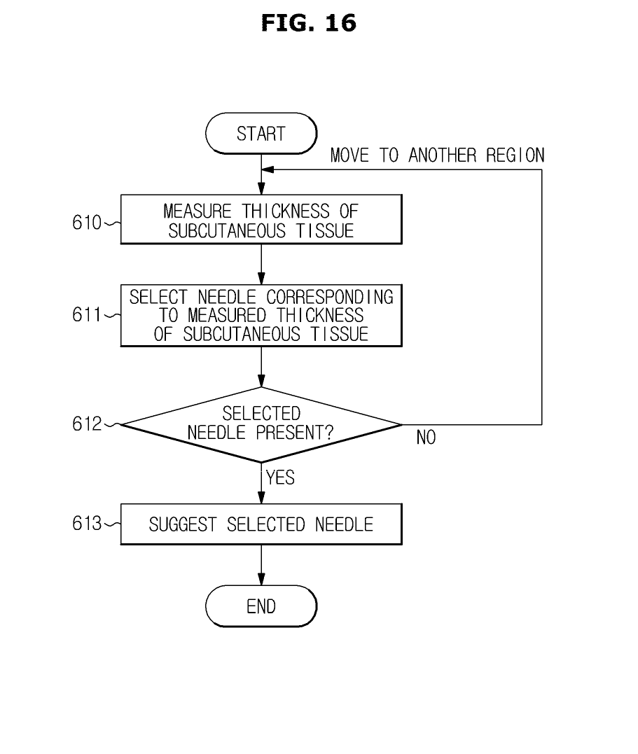

FIG. 16 is a flowchart showing a control method of the ultrasound apparatus including selection of needle length.

Referring to FIG. 16, the thickness of subcutaneous tissue in a region of the subject upon whom injection is to be performed is measured (610). To measure the thickness of subcutaneous tissue, transmission of ultrasound to the injection candidate region, reception of ultrasound echo reflected from the interior of the subject, and various forms of signal processing are performed to acquire ultrasound data required for thickness measurement. Then, the thickness of subcutaneous tissue is calculated using the acquired ultrasound data. Calculation of the thickness of subcutaneous tissue is equal to the above description, and thus a detailed description thereof will be omitted herein.

A needle corresponding to the measured thickness of subcutaneous tissue is selected (611). The selected needle has a length corresponding to the calculated thickness of subcutaneous tissue. The needle length corresponding to the thickness of subcutaneous tissue may differ according to injection methods. A needle may be selected based on an injection method, only a needle suitable for an injection method selected by a user may be selected, or a needle may be selected based on injection using an upright needle in a state in which the skin is not pulled away from the body.

If the selected needle is present (Yes of 612), the selected needle is suggested (613). Suggestion of the selected needle may be visibly performed via the display device provided in the ultrasound apparatus, or may be audibly performed via the speaker provided in the ultrasound apparatus. In an exemplary embodiment, the suggestion is made to a person such as the patient, user, or healthcare professional.

An injection needle has a standard length. Thus, if subcutaneous tissue is excessively thin or thick, a corresponding needle may not be present. That is, if the selected needle is not present (No of 612), the user may be informed of such and may move a probe to another injection candidate region to repeat measurement of the thickness of subcutaneous tissue and needle selection.

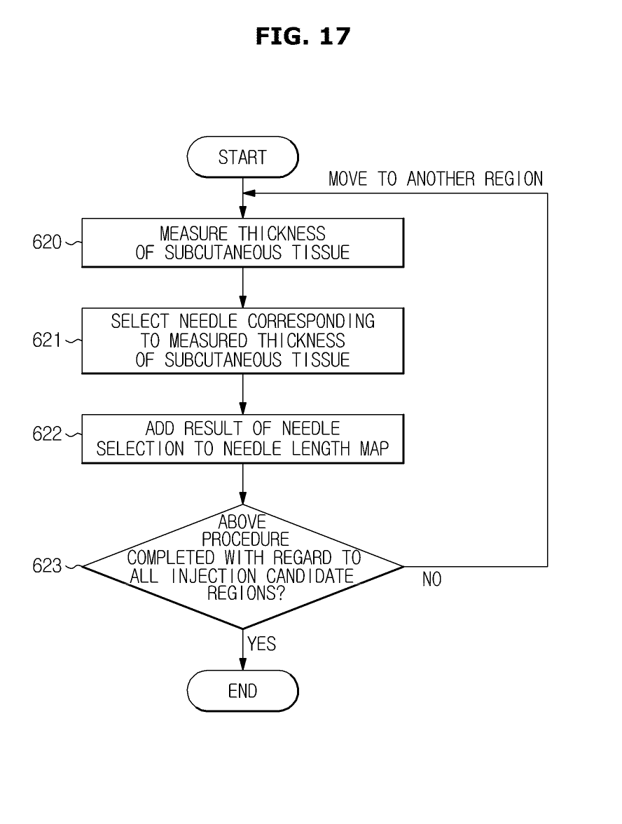

FIG. 17 is a flowchart showing a control method of the ultrasound apparatus including generation of a needle length map.

Referring to FIG. 17, the thickness of subcutaneous tissue is measured (620), and a needle corresponding to the measured thickness of subcutaneous tissue is selected (621). Measurement of the thickness of subcutaneous tissue and needle selection are equal to those in the above-described exemplary embodiment.

Then, the needle selection result is added to a needle length map (622). More specifically, the needle length map matches a needle length selected for an injection candidate region of the subject to a corresponding region. Addition of the needle selection result to the needle length map may be performed by marking the selected needle length on the corresponding region of the needle length map.

Once measurement of the thickness of subcutaneous tissue, selection of the needle, and addition of the needle selection result to the needle length map are completed with regard to all injection candidate regions (Yes of 623), generation of the needle length map ends. If measurement of the thickness of subcutaneous tissue, selection of the needle, and addition of the needle selection result to the needle length map are not completed with regard to all injection candidate regions (No of 623), the probe is moved to another injection candidate region, and measurement of the thickness of subcutaneous tissue, selection of the needle, and addition of the needle selection result to the needle length map are repeated. If insulin is to be injected, certain regions, such as the arm, abdomen, thigh, and hip, may be selected as injection candidate regions.

In the needle length map obtained as described above, as exemplarily shown in FIG. 6, the selected needle length may be marked on the injection candidate region of the subject body. The needle length map may be provided to the patient for self-administration. The body of the subject used in the needle length map may be a real photograph of the subject, or may be an avatar or a representative image corresponding to the subject. In the former case, a camera may be used to capture an image of the subject, and a selected needle length for each insulin injection candidate region may be marked on the captured image of the subject to generate the needle length map.

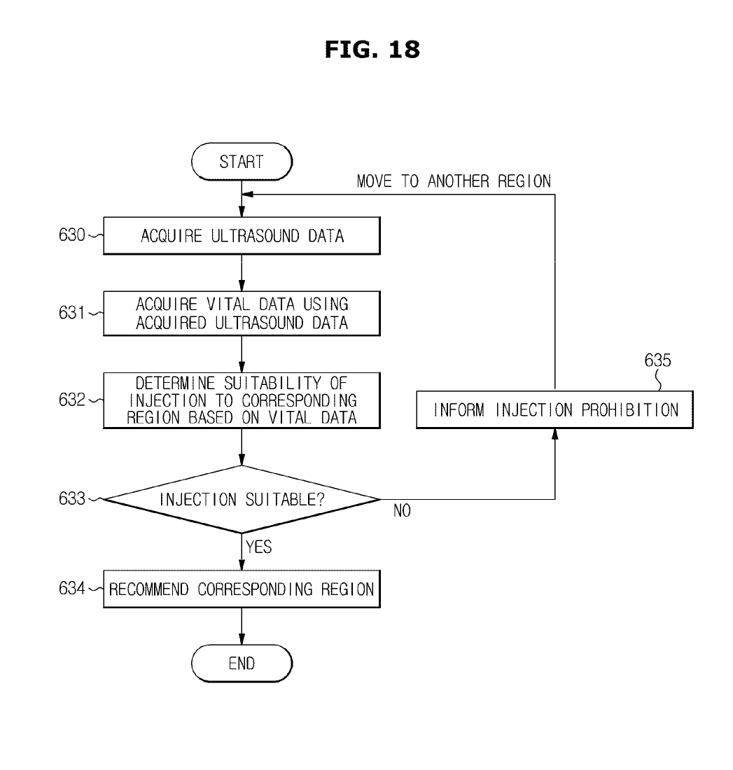

FIG. 18 is a flowchart showing a control method of the ultrasound apparatus including determination of injection suitability.

First, ultrasound data regarding the subject is acquired (630). Vital data regarding the subject is acquired using the acquired ultrasound data (631). Here, the vital data regarding the subject is used to determine suitability of injection to a corresponding region, and may include at least one selected from the group including hardness of the skin, the presence of damaged blood vessels, the presence of inflamed tissue, the presence of varicose veins, and the like.

Ultrasound elasticity imaging and ultrasound Doppler methods may be used to acquire the vital data, and a detailed description thereof has been described with reference to FIG. 8A and thus will be omitted hereinafter.

It is determined whether or not injection to the corresponding region is suitable based on the vital data (632). In one example, if hardness of a corresponding region reaches or exceeds a reference value, the corresponding region may be determined as an insulin-injection prohibition region. In addition, if damaged blood vessels, inflamed tissue or varicose veins are present in a corresponding region, the corresponding region may likewise be determined as an insulin-injection prohibition region.

If the determined result shows that the corresponding region is suitable for injection (Yes of 633), the corresponding region is recommended (634). If the determined result shows that the corresponding region is not suitable for injection (No of 633), the user is informed of prohibition of injection to the corresponding region (635). Then, the user moves the probe to another injection candidate region to repeat the above-described procedure. Injection prohibition may be visibly informed via the display device of the ultrasound apparatus or may be audibly informed via the speaker of the ultrasound apparatus.

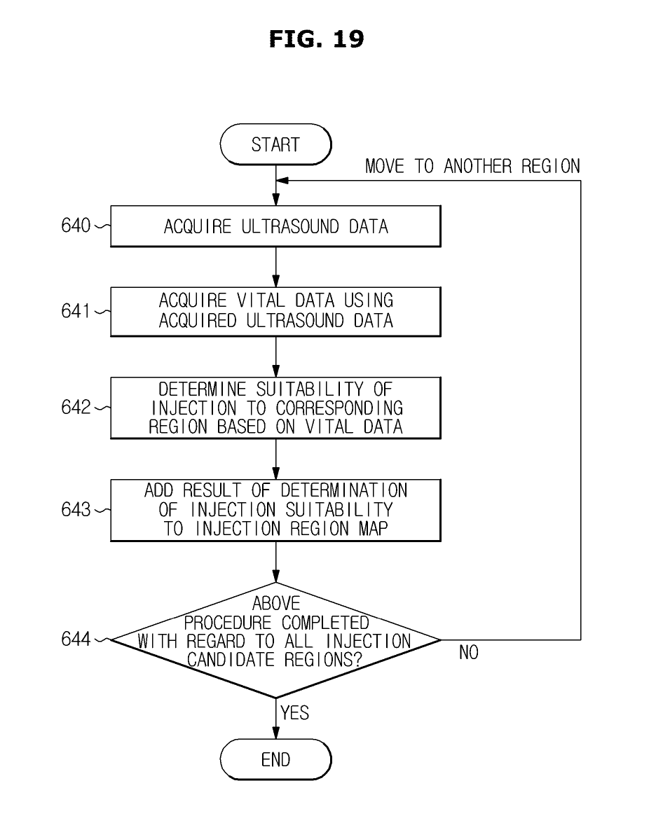

FIG. 19 is a flowchart showing a control method of the ultrasound apparatus including generation of an injection region map.

Referring to FIG. 19, first, ultrasound data regarding the subject is acquired (640), and then vital data regarding the subject is acquired using the acquired ultrasound data (641). Then, it is determined whether or not injection to the corresponding region is suitable based on the vital data (642).

Then, the result of determination of injection suitability is added to an injection region map (643). Injection suitability for each insulin injection candidate region of the subject body is marked on the injection region map. Addition of the result of determination of injection suitability to the injection region map may be performed by marking the result of determination of injection suitability on the corresponding region of the subject body.

Here, the body of the subject may be a real photograph of the subject, or may be an avatar or a representative image corresponding to the subject. In the former case, a camera may be used to capture an image of the subject, and injection suitability for each insulin injection candidate region is marked on the captured image of the subject to generate the injection region map.

If the above procedure is completed with regard to all injection candidate regions (Yes of 644), generation of the injection region map ends. If the above procedure is not completed with regard to all injection candidate regions (No of 644), the user moves the probe to another region to repeat the above procedure.

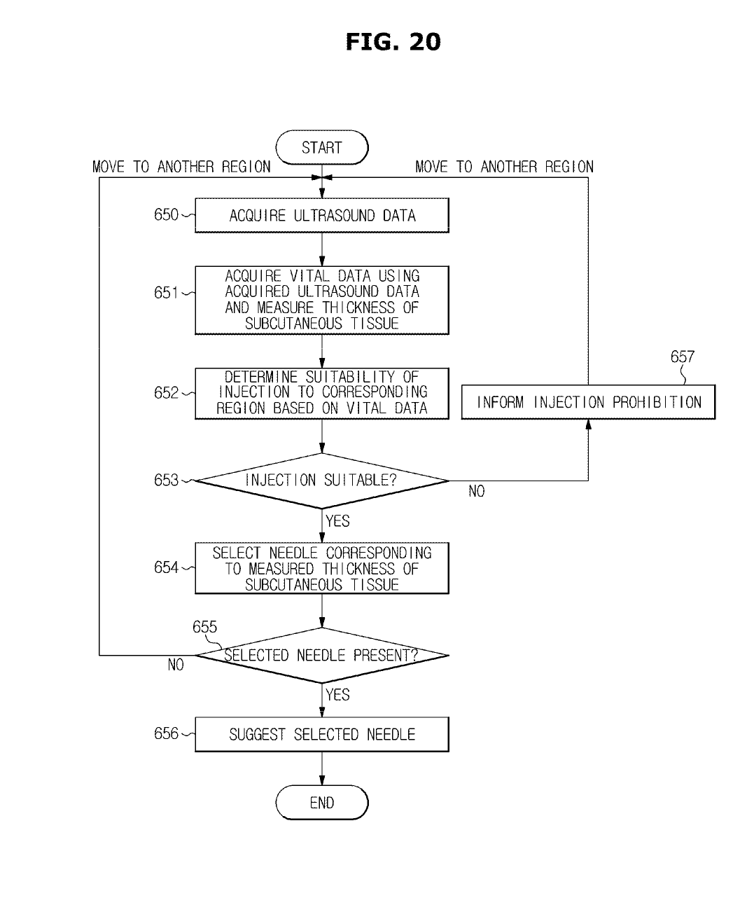

FIG. 20 is a flowchart showing a control method of the ultrasound apparatus including determination of injection suitability and selection of needle length.

Referring to FIG. 20, first, ultrasound data regarding the subject is acquired (650), and then vital data regarding the subject is acquired using the acquired ultrasound data and the thickness of subcutaneous tissue is measured (651). The acquired vital data may be at least one selected from the group including hardness of the skin, the presence of damaged blood vessels, the presence of inflamed tissue, the presence of varicose veins, and the like.

It is determined whether or not injection to a corresponding region is suitable based on the vital data (652). For example, if hardness of a corresponding region reaches or exceeds a reference value, the corresponding region may be determined as an insulin-injection prohibition region. In addition, if damaged blood vessels, inflamed tissue or varicose veins are present in a corresponding region, the corresponding region may likewise be determined as an insulin-injection prohibition region.

If it is determined that injection to the corresponding region is suitable (Yes of 653), a needle corresponding to the measured thickness of subcutaneous tissue is selected (654). The selected needle has a length corresponding to the calculated thickness of subcutaneous tissue, and a description of needle selection is equal to that of the above-described embodiment.

If the selected needle is present (Yes of 655), the selected needle is suggested (656). Suggestion of the selected needle may be visibly performed via the display device provided in the ultrasound apparatus, or may be audibly performed via the speaker provided in the ultrasound apparatus. In an exemplary embodiment, the suggestion is made to a person such as the patient, user, or healthcare professional.

If the selected needle is not present (No of 655), the probe is moved to another region to repeat the above procedure. To allow the user to move the probe to another region, the user may be informed of the absence of the selected needle.

If the determined result shows that the corresponding region is not suitable for injection (No of 653), the user is informed of prohibition of injection to the corresponding region (657). Then, the user moves the probe to another injection candidate region to repeat the above-described procedure from acquisition of ultrasound data to determination of injection suitability. The absence of the selected needle and injection prohibition may be visibly informed via the display device or the speaker.

Meanwhile, although the flowchart of FIG. 20 exemplifies that measurement of the thickness of subcutaneous tissue is performed before determination of suitability of injection to the corresponding region, measurement of the thickness of subcutaneous tissue may be performed after it is determined that the corresponding region is suitable for injection.

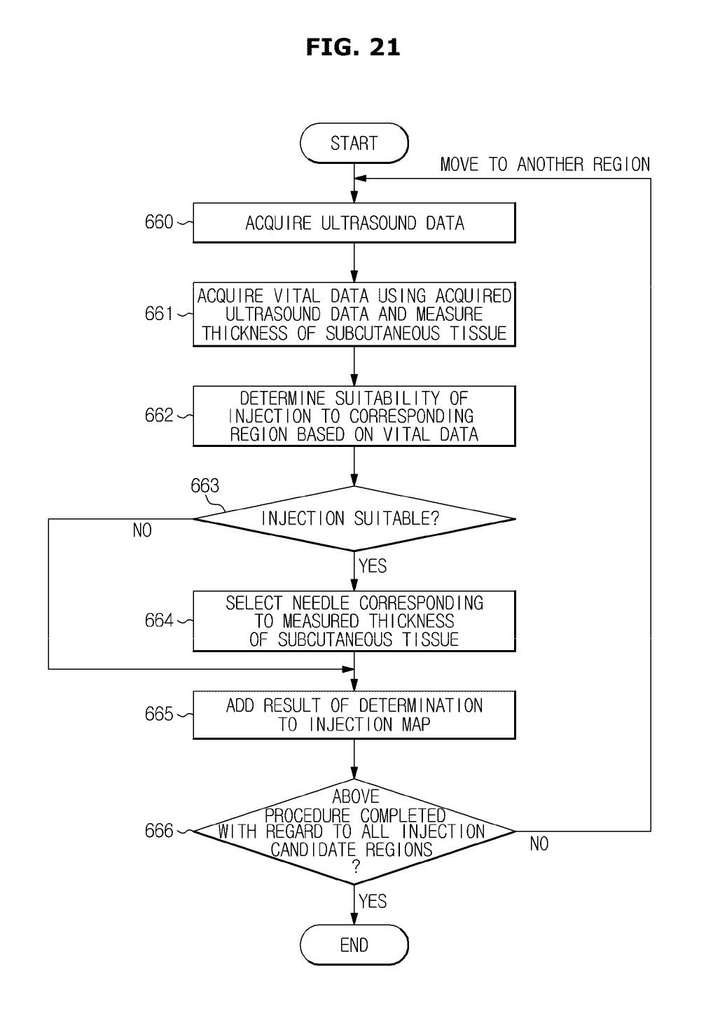

FIG. 21 is a flowchart showing a control method of the ultrasound apparatus including generation of an injection map containing data regarding injection suitability as well as data regarding needle length.

Referring to FIG. 21, first, ultrasound data regarding the subject is acquired (660), and then vital data regarding the subject is acquired using the acquired ultrasound data and the thickness of subcutaneous tissue is measured (661). The acquired vital data may be at least one selected from the group including hardness of the skin, the presence of damaged blood vessels, the presence of inflamed tissue, the presence of varicose veins, and the like.

It is determined whether or not injection to a corresponding region is suitable based on the vital data (662). For example, if hardness of a corresponding region reaches or exceeds a reference value, the corresponding region may be determined as an insulin-injection prohibition region. In addition, if damaged blood vessels, inflamed tissue or varicose veins are present in a corresponding region, the corresponding region may likewise be determined as an insulin-injection prohibition region.

If it is determined that injection to the corresponding region is suitable (Yes of 663), a needle corresponding to the measured thickness of subcutaneous tissue is selected (664). The selected needle has a length corresponding to the calculated thickness of subcutaneous tissue, and a description of needle selection is equal to that of the above-described embodiment.

The needle selection result is added to an injection map (665). The injection map indicates suitability of injection to injection candidate regions as well as a corresponding needle length. In the related art, the needle selection result, i.e. a selected needle length is marked on a corresponding region of the injection map.

If it is determined that injection to the corresponding region is not suitable (No of 663), the result of determination is added to the injection map (665). That is, an injection prohibition mark is provided in the corresponding region of the injection map. This mark assists the user in recognizing injection prohibition and is not limited to a specific form.

If the above procedure is completed with regard to all injection candidate regions (Yes of 666), generation of the injection map ends. If the above procedure is not completed with regard to all injection candidate regions (No of 666), the probe is moved to another region to repeat the above procedure.

The generated injection map may be provided the patient in the form of a file or other output formats, and the patient or patient's caretaker may perform self-administration using the injection map.

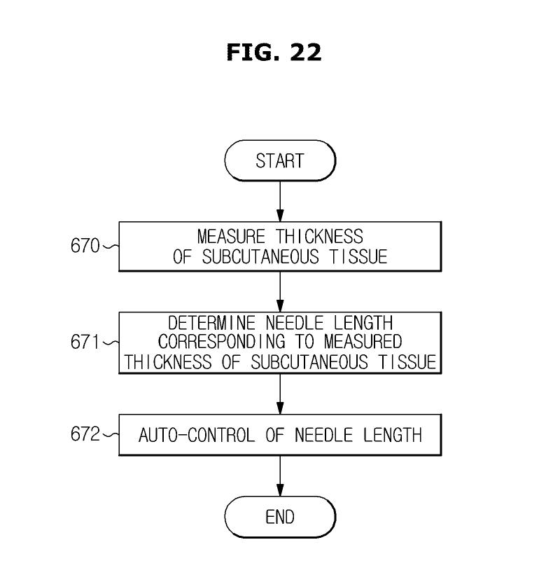

FIG. 22 is a flowchart showing a control method of the ultrasound apparatus including auto-control of needle length.

Referring to FIG. 22, first, the thickness of subcutaneous tissue of the subject is measured (670). Measurement of the thickness of subcutaneous tissue has been described in the above exemplary embodiment, and a description thereof will be omitted hereinafter.

A needle length corresponding to the measured thickness of subcutaneous tissue is determined (671). The needle length corresponding to the measured thickness of subcutaneous tissue is a length ensuring that a needle tip is inserted into subcutaneous tissue located between the skin and muscles.

Then, the needle length is automatically controlled (672). To this end, the probe module equipped with the syringe may be used. One example of the probe module is shown in FIGS. 15 and 16. Control of needle length may be performed by controlling an insertion depth of a needle into the subject. For example, if the determined needle length is 5 mm, the needle of the syringe mounted in the probe module is controlled so as to be inserted by 5 mm.

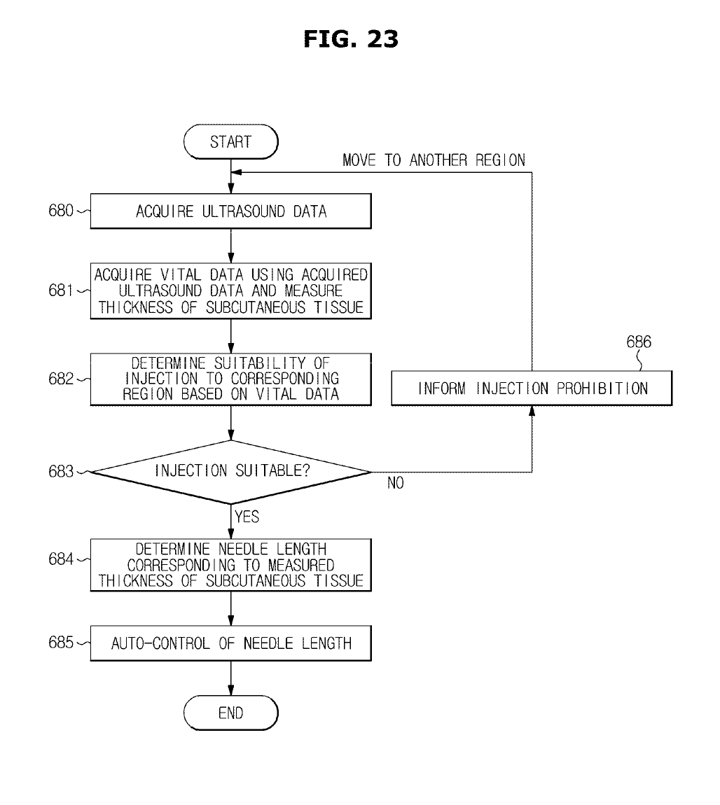

FIG. 23 is a flowchart showing a control method of the ultrasound apparatus including determination of injection suitability and auto-control of needle length.

Referring to FIG. 23, first, ultrasound data regarding the subject is acquired (680). Then, vital data regarding the subject is acquired using the acquired ultrasound data and the thickness of subcutaneous tissue is measured (681). The acquired vital data may be at least one selected from the group including hardness of the skin, the presence of damaged blood vessels, the presence of inflamed tissue, the presence of varicose veins, and the like, but the exemplary embodiment is not limited thereto. The vital data of the present exemplary embodiment may include other data so long as such data aids in determination of injection candidate regions.

Then, it is determined whether or not injection to a corresponding region is suitable based on the acquired vital data (682). For example, if hardness of a corresponding region reaches or exceeds a reference value, the corresponding region may be determined as an insulin-injection prohibition region. In addition, if damaged blood vessels, inflamed tissue or varicose veins are present in a corresponding region, the corresponding region may likewise be determined as an insulin-injection prohibition region.

If it is determined that injection to the corresponding region is suitable (Yes of 683), a needle length corresponding to the measured thickness of subcutaneous tissue is determined (684), and the needle length is automatically controlled using the syringe mounted in the probe module (685). Auto-control of the needle length has been described in the above exemplary embodiment.

If it is determined that injection to the corresponding region is not suitable (No of 683), the user is informed of prohibition of injection to the corresponding region (686) to allow the user to move the probe module to another region. Then, the procedure from acquisition of ultrasound data to determination of injection suitability is repeated.

Meanwhile, although the flowchart of FIG. 23 exemplifies that measurement of the thickness of subcutaneous tissue is performed before determination of suitability of injection to the corresponding region, measurement of the thickness of subcutaneous tissue may be performed only after it is determined that the corresponding region is suitable for injection.

Although the above exemplary embodiments describe the ultrasound apparatus and the control method thereof as being applied to insulin injection, the exemplary embodiments may be applied to injection of other drugs except for insulin. In the case of intra-dermal injection or intramuscular injection rather than subcutaneous injection, the thickness calculated by the thickness calculator 120, 220, 320 or 420 may be the thickness of the skin or muscles rather than subcutaneous tissue.

As is apparent from the above description, with an ultrasound apparatus and a control method thereof according to the above-described embodiments, the thickness of subcutaneous tissue may be measured using an ultrasound probe or vital data regarding a subject may be acquired to select an appropriate injection needle and determine suitability of injection to a corresponding body region, which ensures safe and effective drug administration.

Further, as a map containing results of selection of an appropriate injection needle and determination of injection suitability is generated and provided to a patient, it may be possible to ensure safe self-administration by the patient without a hospital visit.

Although the exemplary embodiments of the present invention have been shown and described, it would be appreciated by those skilled in the art that changes may be made in these embodiments without departing from the principles and spirit of the invention, the scope of which is defined in the claims and their equivalents.

What is claimed is:

1. An ultrasound apparatus comprising:
a data acquirer configured to acquire ultrasound data regarding a subject;
a thickness calculator configured to calculate a thickness of subcutaneous tissue of the subject based on the ultrasound data; and
a needle selector configured to select a needle based on the calculated thickness of subcutaneous tissue.

2. The apparatus according to claim 1, wherein the data acquirer comprises an ultrasound probe configured to transmit ultrasound to the subject and receive ultrasound reflected from the subject.

3. The apparatus according to claim 2, wherein the selected needle comprises a length corresponding to the calculated thickness of subcutaneous tissue.

4. The apparatus according to claim 3, wherein the length is greater than a depth of a skin, to ensure insertion of a needle tip into the subcutaneous tissue.

5. The apparatus according to claim 3, further comprising at least one from among a display device and a speaker to provide at least one from among a visual output and an audible output corresponding to the selected needle.

6. The apparatus according to claim 3, further comprising a map generator configured to generate a needle length map by matching the needle selected for a region of the subject.

7. The apparatus according to claim 3, further comprising:
a vital data acquirer configured to acquire vital data regarding the subject based on the ultrasound data; and
an injection region determiner configured to determine injection suitability based on the acquired vital data regarding the subject.

8. The apparatus according to claim 7, wherein the vital data regarding the subject is at least one selected from the group including hardness of the skin, presence of varicose veins, and presence of damaged blood vessels.

9. The apparatus according to claim 8, wherein the injection region determiner determines that injection is not suitable if the hardness of the skin reaches or exceeds a predetermined reference value, if varicose veins are present, or if damaged blood vessels are present.

10. The apparatus according to claim 7, further comprising at least one from among a display device and a speaker configured to output the determined injection suitability.

11. The apparatus according to claim 7, further comprising a map generator configured to generate an injection map by matching the selected needle and the determined injection suitability for a region of the subject.

12. The apparatus according to any one of claim 3, further comprising:
a syringe coupled to the ultrasound probe; and
a syringe controller configured to control a needle length of the syringe according to the needle selected by the needle selector.

13. An ultrasound apparatus comprising:
a data acquirer configured to acquire ultrasound data regarding a subject;
a vital data acquirer configured to acquire vital data regarding the subject based on the ultrasound data; and
an injection region determiner configured to determine injection suitability based on the acquired vital data regarding the subject.

14. The apparatus according to claim 13, wherein the vital data regarding the subject is at least one selected from the group including hardness of the skin, presence of varicose veins, and presence of damaged blood vessels.

15. The apparatus according to claim 14, wherein the injection region determiner determines that injection is not suitable if hardness of the skin reaches or exceeds a predetermined reference value, if varicose veins are present, or if damaged blood vessels are present.

16. The apparatus according to claim 13, further comprising at least one from among a display device and a speaker configured to output the determined injection suitability.

17. The apparatus according to claim 12, further comprising a map generator configured to generate an injection region map by matching the determined injection suitability for a region of the subject.

18. The apparatus according to claim 3, further comprising:
a syringe coupled to the probe; and
a syringe controller configured to control the syringe such that a needle of the syringe is inserted into the subject according to the length of the selected needle.

19. A control method of an ultrasound apparatus, comprising:
acquiring ultrasound data regarding a subject;
measuring thickness of subcutaneous tissue of the subject based on the acquired ultrasound data; and
selecting a needle corresponding to the measured thickness of subcutaneous tissue.

20. The method according to claim 19, wherein the selecting the needle comprises selecting the needle having a length corresponding to the measured thickness of subcutaneous tissue.

21. The method according to claim 20, wherein the length is greater than a depth of a skin to ensure insertion of a needle tip into the subcutaneous tissue.

22. The method according to claim 20, further comprising generating a needle length map by matching the selected needle for a region of the subject.

23. The method according to claim 20, further comprising:
acquiring vital data regarding the subject based on the acquired ultrasound data; and
determining suitability of injection to a corresponding region based on the acquired vital data regarding the subject.

24. The method according to claim 23, wherein the vital data regarding the subject is at least one selected from the group including hardness of the skin, presence of varicose veins, and presence of damaged blood vessels.

25. The method according to claim 24, wherein the determining the suitability of injection comprises determining that injection is not suitable if hardness of the skin reaches or exceeds a predetermined reference value, if varicose veins are present, or if damaged blood vessels are present.

26. The method according to claim 23, further comprising visibly or audibly outputting a result of the determining the suitability of injection.

27. The method according to claim 23, further comprising generating an injection map by matching the selected needle and a result of the determining the suitability of injection for a region of the subject.

28. The method according to any one of claims 20, further comprising automatically controlling a needle length of the syringe according to the length of the selected needle.

29. The method according to any one of claims 20, further comprising outputting at least one from among a visual output and an audible output corresponding to the selected needle.

30. A control method of an ultrasound apparatus, comprising:
acquiring ultrasound data regarding a subject;
acquiring vital data regarding the subject based on the acquired ultrasound data; and
determining suitability of injection to a corresponding region based on the acquired vital data.

31. The method according to claim 30, wherein the vital data regarding the subject is at least one selected from the group including hardness of the skin, presence of varicose veins, and presence of damaged blood vessels.

32. The method according to claim 31, wherein determination of injection suitability includes determining that injection is not suitable if hardness of the skin reaches or exceeds a predetermined reference value, if varicose veins are present, or if damaged blood vessels are present.

33. The method according to claim 30, further comprising visibly or audibly outputting a result of the determining of suitability of injection.
